(19) United States
(12) Patent Application Publication
(10) Pub. No.: US 2006/0009497 A1
(43) Pub. Date: Jan. 12, 2006

(54) Novel tetrahydropyridine derivatives as renin inhibitors

(75) Inventors: Olivier Bezencon, Allschwil (CH); Daniel Bur, Therwil (CH); Walter Fischli, Allschwil (CH); Lubos Remen, Allschwil (CH); Sylvia Richard-Bildstein, Dietwiller (FR); Thomas Weller, Binningen (CH)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(73) Assignee: ACTELION PHARMACEUTICALS, LTD., Allschwil (CH)

(21) Appl. No.: 10/514,164

(22) PCT Filed: Apr. 29, 2003
(86) PCT No.: PCT/EP03/04445

§ 371 Date: Nov. 12, 2004

(30) Foreign Application Priority Data
Jun. 27, 2002 (WO) PCT/EP02/07102

(51) Int. Cl. C07D 211/82; A61K 31/455

(52) U.S. Cl. 514/355; 546/315

(57) ABSTRACT

The invention relates to novel tetrahydropyridine derivatives and related compounds and their use as active ingredients in the preparation of pharmaceutical compositions. The invention also concerns related aspects including processes for the preparation of the compounds, pharmaceutical compositions containing one or more of those compounds and especially their use as inhibitors of renin.

The invention relates to novel compounds of the general formula I. The invention also concerns related aspects including processes for the preparation of the compounds, pharmaceutical compositions containing one or more compounds of formula I and especially their use as renin inhibitors in cardiovascular events and renal insufficiency. Furthermore, some of these compounds can be regarded as inhibitors of other aspartyl proteases and might therefore be useful as inhibitors of plasmepsins to treat malaria and as inhibitors of Candida albicans secreted aspartyl proteases to treat fungal infections.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes to Ang II, whose concentration is dramatically increased by the blockade of AT₁ receptors. This may raise serious questions regarding the safety and efficacy profile of AT₁ receptor antagonists. In summary, renin inhibitors are not only expected to be different from ACE inhibitors and AT₁ blockers with regard to safety, but more importantly also with regard to their efficacy to block the RAS.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are missing and sought. Recently, the first non-peptide renin inhibitors were described which show high in vitro activity (Oefner C. et al., Chem. Biol., 1999, 6, 127; Patent Application WO97/093 11; Märki H. P. et al., II Farmaco, 2001, 56, 21). However, the development status of these compounds is not known.

The present invention relates to the unexpected identification of renin inhibitors of a non-peptidic nature and of low molecular weight. Orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis, are described.

In particular, the present invention relates to novel compounds of the general formula I. 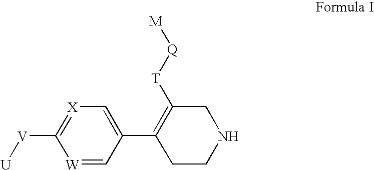
wherein
X and W represent independently a nitrogen atom or a CH-group;
V represents —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; —CH₂—CH₂-A-CH₂—CH₂—B—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; —COO—; —(CH₂)ₚOCONR¹—; —(CH₂)ₚN(R¹′)CONR¹—;
Q represents lower alkylene; lower alkenylene;
M represents hydrogen; cycloalkyl; aryl; heterocyclyl; heteroaryl;
R¹ and R¹′ independently represent hydrogen; lower alkyl; lower alkenyl; lower alkinyl; cycloalkyl; aryl; cycloalkyl-lower alkyl;
p is the integer 1, 2, 3 or 4;
r is the integer 3, 4, 5 or 6;
s is the integer 2, 3, 4 or 5;
t is the integer 1, 2, 3 or 4;
u is the integer 1, 2 or 3;
v the integer to 2, 3 or 4;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

In the definitions of general formula I—if not otherwise stated—the term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

The term lower alkoxy refers to a R—O group, wherein R is a lower alkyl. Examples of lower alkoxy groups are methoxy, ethoxy, propoxy, iso-propoxy, iso-butoxy, sec-butoxy and tert-butoxy.

The term lower alkenyl, alone or in combination with other groups, means straight and branched chain groups comprising an olefinic bond and two to seven carbon atoms, preferably two to four carbon atoms, that can be optionally substituted by halogens. Examples of lower alkenyl are vinyl, propenyl or butenyl.

The term lower alkinyl, alone or in combination with other groups, means straight and branched chain groups comprising a triple bond and two to seven carbon atoms, preferably two to four carbon atoms, that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms, that can be optionally substituted by halogens. Examples of lower alkylene are ethylene, propylene or butylene.

The term lower alkenylene, alone or in combination with other groups, means straight and branched divalent chain groups comprising an olefinic bond and two to seven carbon atoms, preferably two to four carbon atoms, that can be optionally substituted by halogens. Examples of lower alkenylene are vinylene, propenylene and butenylene.

The term lower alkylenedioxy, refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono-, di-, or trisubstituted independently by lower alkyl, lower alkenyl, lower alkenylene, lower alkoxy, lower alkylenoxy, lower alkylenedioxy, hydroxy, halogen, —CF₃, —NR¹R¹′, —NR¹C(O)R¹′, —NR¹S(O)₂R¹′, —C(O)NR¹R¹′, lower alkylcarbonyl, —COOR¹, —SR¹, —SOR¹, —SO₂R¹, —SO₂NR¹R¹¹. The cyclopropyl group is a preferred group.

The term aryl, alone or in combination, relates to the phenyl, the naphthyl or the indanyl group, preferably the phenyl group, which can be optionally mono-, di-, tri-, tetra- or pentasubstituted independently by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹R¹′, —NR¹R¹′-lower alkyl, —NR¹C(O)R¹′, —NR₁S(O)₂R¹′, —C(O)NR¹R¹′, —NO₂, lower alkylcarbonyl, —COOR¹, —SR¹, —S(O)R¹, —S(O)₂R¹, —SO₂NR¹R¹′, benzyloxy. Preferred substituents are halogen, lower alkoxy, lower alkyl.

The term aryloxy refers to an Ar—O group, wherein Ar is an aryl. An example of aryloxy groups is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a COOR² group. Examples of such rings are piperidinyl, morpholinyl, thiomorpholinyl, piperazinyl, tetrahydropyranyl, dihydropyranyl, 1,4-dioxanyl, pyrrolidinyl, tetrahydrofuranyl, dihydropyrrolyl, imidazolidinyl, dihydropyrazolyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequatly substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R¹′, NR¹R¹′-lower alkyl, —N(R¹)COR¹, —N(R¹)SO₂R¹, —CONR¹R¹′, —NO₂, lower alkylcarbonyl, —COOR¹, —SR¹, —S(O)R¹, —S(O)₂R¹, —SO₂NR¹R¹′, another aryl, another heteroaryl or another heterocyclyl and the like.

The term heteroaryloxy refers to a Het-O group, wherein Het is a heteroaryl.

It is understoood that the substituents outlined relative to the expressions cycloalkyl, heterocyclyl, heteroaryl and aryl have been omitted in the definitions of the general formula I and in claims 1 to 6 for clarity reasons but the definitions in formula I and in claims 1 to 6 should be read as if they are included therein.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula I is acidic in nature with an inorganic base like an alkali or earth alkali base, e.g. sodium hydroxide, potassium hydroxide, calcium hydroxide and the like.

The compounds of the general formula I can contain one or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts therof.

The present invention encompasses all these forms. Mixtures may be separated in a manner known per se, i.e. by column chromatography, thin layer chromatography, HPLC or crystallization.

A group of preferred compounds of general formula I are those wherein X, W, V, and U, are as defined in general formula I and wherein
T is —CONR¹—;
Q is a methylene;
M is hydrogen; aryl; heteroaryl.

Another group of more preferred compounds of general formula I are those wherein X, W, T, Q, and M are as defined in general formula I and wherein
V is one of the following groups:
—CH₂CH₂O—; —CH₂CH₂CH₂O—; —OCH₂CH₂O—
and U is as defined in general formula I above.

Another group of even more preferred compounds of general formula I are those wherein V, U, T, Q, and M are as defined in general formula I and wherein
X and W represent CH.

Another group of more preferred compounds of general formula I are those wherein X, W, V, Q, T, and M are as defined in general formula I and wherein
U is a mono-, di-, or trisubstituted phenyl. Preferred substituents are independently halogen or lower alkyl, lower alkoxy, trifluoromethyl, trifluoromethoxy.

Especially preferred compounds of general formula I are those selected from the group consisting of:
4-{4-[3-(2-methoxybenzyloxy)propoxy]phenyl}-1,2,5,6-tetrahydropyridine-3-carboxylic acid [2-(2-chlorophenyl)ethyl]methylamide,
4-{4-[3-(2-bromo-5-fluorophenoxy)propyl]phenyl-1,2,5,6-tetrahydropyridi-ne-3-carboxylic acid [2-(2-chlorophenyl)ethyl]methylamide,
4-{4-[3-(2-bromo-5-fluorophenoxy)propyl]phenyl}-1,2,5,6-tetrahydropyridi-ne-3-carboxylic acid 2-phenethylmethylamide,
4-{4-[3-(2-bromo-5-fluorophenoxy)propyl]phenyl}-1,2,5,6-tetrahydropyridi-ne-3-carboxylic acid (2-chlorobenzyl)methylamide,
4-{4-[3-(2-bromo-5-fluorophenoxy)propyl]phenyl}-1,2,5,6-tetrahydropyridi-ne-3-carboxylic acid (2-chlorobenzyl)cyclopropylamide,
4-{4-[3-(2-chlorophenoxy)propyl]phenyl}-1,2,5,6-tetrahydropyridine-3-carboxylic acid [2-(2-chlorophenyl)ethyl]methylamide,
4-{4-[3-(2-chlorophenoxy)propyl]phenyl}-1,2,5,6-tetrahydropyridine-3-carboxylic acid 2-phenethylmethylamide,
4-{4-[3-(2-chlorophenoxy)propyl]phenyl}-1,2,5,6-tetrahydropyridine-3-carboxylic acid (2-chlorobenzyl)methylamide,
4-{4-[3-(2-chlorophenoxy)propyl]phenyl}-1,2,5,6-tetrahydropyridine-3-carboxylic acid (2-chlorobenzyl)cyclopropylamide,
4-{4-[3-(2,5-difluorophenoxy)propyl]phenyl}-1,2,5,6-tetrahydropyridine-3-carboxylic acid [2-(2-chlorophenyl)ethyl]methylamide,
4-{4-[3-(2,5-difluorophenoxy)propyl]phenyl}-1,2,5,6-tetrahydropyridine-3-carboxylic acid 2-phenethylmethylamide,
4-{4-[3-(2,5-difluorophenoxy)propyl]phenyl}-1,2,5,6-tetrahydropyridine-3-carboxylic acid (2-chlorobenzyl)methylamide,
4-{4-[3-(2,5-difluorophenoxy)propyl]phenyl}-1,2,5,6-tetrahydropyridine-3-carboxylic acid (2-chlorobenzyl)cyclopropylamide,
4-{4-[3-(2-bromo-5-fluorophenoxy)propyl]phenyl}-1,2,5,6-tetrahydro-pyridine-3-carboxylic acid (2-chlorobenzyl)cyclopropylamide,
4-{4-[3-(2,3,6-trifluorophenoxy)propyl]phenyl}-1,2,5,6-tetrahydropyridine-3-carboxylic acid (2-chlorobenzyl)cyclopropylamide,
4-{4-[3-(2,3,6-trifluorophenoxy)propyl]phenyl}-1,2,5,6-tetrahydropyridine-3-carboxylic acid (2-chlorobenzyl)ethylamide,
4-{4-[3-(2,3,6-trifluorophenoxy)propyl]phenyl}-1,2,5,6-tetrahydropyridine-3-carboxylic acid cyclopropyl-(2-fluorobenzyl)amide,
4-{4-[3-(2,3,6-trifluorophenoxy)propyl]phenyl}-1,2,5,6-tetrahydropyridine-3-carboxylic acid cyclopropyl-(3-trifluoromethylbenzyl)amide,
4-{4-[3-(2,3,6-trifluorophenoxy)propyl]phenyl}-1,2,5,6-tetrahydropyridine-3-carboxylic acid cyclopropyl-(2-methylbenzyl)amide,
4-{4-[3-(2,3,6-trifluorophenoxy)propyl]phenyl}-1,2,5,6-tetrahydropyridine-3-carboxylic acid cyclopropyl-[2-(4-methoxyphenoxy)ethyl]amide,
4-{4-[3-(2,3,6-trifluorophenoxy)propyl]phenyl}-1,2,5,6-tetrahydropyridine-3-carboxylic acid cyclopropyl-[2-(3-methoxyphenoxy)ethyl]amide,
4-{4-[3-(2,3,6-trifluorophenoxy)propyl]phenyl}-1,2,5,6-tetrahydropyridine-3-carboxylic acid cyclopropyl-(2-m-tolyloxyethyl)amide,
4-{4-[3-(2,3,6-trifluorophenoxy)propyl]phenyl}-1,2,5,6-tetrahydropyridine-3-carboxylic acid [2-(2-chlorophenyl)ethyl]cyclopropylamide,
4-{4-[3-(2,3,6-trifluorophenoxy)propyl]phenyl}-1,2,5,6-tetrahydropyridine-3-carboxylic acid cyclopropyl-[2-(4-fluorophenyl)ethyl]amide,
4-{4-[3-(2,3,6-trifluorophenoxy)propyl]phenyl}-1,2,5,6-tetrahydropyridine-3-carboxylic and acid cyclopropyl-(2-o-tolylethyl)amide,
4-{4-[3-(2,3,6-trifluorophenoxy)propyl]phenyl}-1,2,5,6-tetrahydropyridine-3-carboxylic acid cyclopropyl-(3,5-dimethoxybenzyl)amide,
4-{4-[3-(2,3,6-trifluorophenoxy)propyl]phenyl}-1,2,5,6-tetrahydropyridine-3-carboxylic acid cyclopropyl-(2-p-tolylethyl)amide,
4-{4-[2-(2,3,5-trimethylphenoxy)ethyl]phenyl}-1,2,5,6-tetrahydropyridine-3-carboxylic acid cyclopropyl-(3-trifluoromethylbenzyl)amide,
4-{4-[2-(2,3,5-trimethylphenoxy)ethyl]phenyl}-1,2,5,6-tetrahydropyridine-3-carboxylic acid cyclopropyl-(2-methylbenzyl)amide,
4-{4-[2-(2,3,5-trimethylphenoxy)ethyl]phenyl}-1,2,5,6-tetrahydropyridine-3-carboxylic acid cyclopropylphenethylamide,
4-{4-[3-(2-bromo-5-fluorophenoxy)propyl]phenyl}-1,2,5,6-tetrahydropyridine-3-carboxylic acid (2-chlorobenzyl)ethylamide,
4-{4-[3-(2-bromo-5-fluorophenoxy)propyl]phenyl}-1,2,5,6-tetrahydropyridine-3-carboxylic acid cyclopropyl-(2-methylbenzyl)amide,
4-{4-[3-(2-bromo-5-fluorophenoxy)propyl]phenyl}-1,2,5,6-tetrahydropyridine-3-carboxylic acid cyclopropyl-[2-(4-methoxyphenoxy)ethyl]amide,
4-{4-[3-(2-bromo-5-fluorophenoxy)propyl]phenyl}-1,2,5,6-tetrahydropyridine-3-carboxylic acid cyclopropylphenethylamide,
4-{4-[3-(2-bromo-5-fluorophenoxy)propyl]phenyl}-1,2,5,6-tetrahydropyridine-3-carboxylic acid cyclopropyl-(2-o-tolylethyl)amide,
4-{4-[3-(2-bromo-5-fluorophenoxy)propyl]phenyl}-1,2,5,6-tetrahydropyridine-3-carboxylic acid cyclopropyl-(3,5-dimethoxybenzyl)amide,
4-{4-[3-(2-bromo-5-fluorophenoxy)propyl]phenyl)-1,2,5,6-tetrahydro-pyridine-3-carboxylic acid cyclopropyl-(2-p-tolylethyl)amide,
4-{4-[2-(2-chloro-4,5-dimethylphenoxy)ethoxy]phenyl}-1,2,5,6-tetrahydro-pyridine-3-carboxylic acid (2-chlorobenzyl)cyclopropylamide,
4-{4-[3-(2,3,6-trifluorophenoxy)propyl]phenyl}-1,2,5,6-tetrahydropyridine-3-carboxylic acid cyclopropyl-(2-fluoro-5-methoxybenzyl)amide,
4-{4-[3-(2,3,6-trifluorophenoxy)propyl]phenyl}-1,2,5,6-tetrahydropyridine-3-carboxylic acid cyclopropyl-(3-methoxybenzyl)amide,
4-{4-[3-(2,3,6-trifluorophenoxy)propyl]phenyl}-1,2,5,6-tetrahydropyridine-3-carboxylic acid cyclopropyl-(3,4-dimethoxybenzyl)amide,
4-f{4-[3-(2,3,6-trifluorophenoxy)propyl]phenyl}-1,2,5,6-tetrahydropyridine-3-carboxylic acid (2-chloro-3-trifluoromethylbenzyl)cyclopropylamide,
4-{4-[3-(2,3,6-trifluorophenoxy)propyl]phenyl}-1,2,5,6-tetrahydropyridine-3-carboxylic acid (6-chlorobenzo[1,3]dioxol-5-ylmethyl)cyclopropylamide,
4-{4-[3-(2,3,6-trifluorophenoxy)propyl]phenyl}-1,2,5,6-tetrahydropyridine-3-carboxylic acid (2-chloro-6-fluorobenzyl)cyclopropylamide,
4-14-[3-(2,3,6-trifluorophenoxy)propyl]phenyl)-1,2,5,6-tetrahydropyridine-3-carboxylic acid (2-bromobenzyl)cyclopropylamide,
4-{4-[3-(2,3,6-trifluorophenoxy)propyl]phenyl}-1,2,5,6-tetrahydropyridine-3-carboxylic acid cyclopropyl-(2,3-dimethylbenzyl)amide,
4-{4-[3-(2,3,6-trifluorophenoxy)propylphenyl}-1,2,5,6-tetrahydropyridine-3-carboxylic acid cyclopropyl-(3-fluoro-2-methylbenzyl)amide,
4-{4-[3-(2,3,6-trifluorophenoxy)propyl]phenyl}-1,2,5,6-tetrahydropyridine-3-carboxylic acid cyclopropyl-(2,3-dichlorobenzyl)amide,
4-{4-[3-(2,3,6-trifluorophenoxy)propyl]phenyl}-1,2,5,6-tetrahydropyridine-3-carboxylic acid cyclopropyl-(3-methylbenzyl)amide,
4-{4-[3-(2,3,6-trifluorophenoxy)propyl]phenyl}-1,2,5,6-tetrahydropyridine-3-carboxylic acid cyclopropyl-(2,3-difluorobenzyl)amide,
4-{4-[3-(2,3,6-trifluorophenoxy)propyl]phenyl)-1,2,5,6-tetrahydropyridine-3-carboxylic acid (3-chlorobenzyl)cyclopropylamide,
4-{4-[2-(2,6-dichloro-4-methylphenoxy)ethoxy]phenyl}-1,2,5,6-tetrahydro-pyridine-3-carboxylic acid cyclopropyl-(2,3-dichlorobenzyl)amide,
4-{4-[2-(2,6-dichloro-4-methylphenoxy)ethoxy]phenyl}-1,2,5,6-tetrahydro-pyridine-3-carboxylic acid (2-chloro-3-trifluoromethylbenzyl)cyclopropylamide,
4-{4-[2-(2,6-dichloro-4-methylphenoxy)ethoxy]phenyl}-1,2,5,6-tetrahydro-pyridine-3-carboxylic acid cyclopropyl-(2,3-dimethylbenzyl)amide,
4-{4-[2-(2,3,6-trifluorophenoxy)ethoxy]phenyl}-1,2,5,6-tetrahydropyridine-3-carboxylic acid (2-chloro-3-trifluoromethylbenzyl)cyclopropylamide,
4-{4-[2-(2,6-dichloro-4-fluorophenoxy)ethoxy]phenyl}-1,2,5,6-tetrahydro-pyridine-3-carboxylic acid (2-bromobenzyl)cyclopropylamide,
4-{4-[2-(2,4,6-trifluorophenoxy)ethoxy]phenyl}-1,2,5,6-tetrahydropyridine-3-carboxylic acid cyclopropyl-(2,3-dichlorobenzyl)amide,
4-{4-[2-(2,6-dichloro-4-fluorophenoxy)ethoxy]phenyl}-1,2,5,6-tetrahydro-pyridine-3-carboxylic acid cyclopropyl-(2,3-dichlorobenzyl)amide,
4-{4-[2-(2-chloro-4,5-dimethylphenoxy)ethoxy]phenyl})1,2,5,6-tetrahydro-pyridine-3-carboxylic acid cyclopropyl-(2,3-dichlorobenzyl)amide,
4-{4-[2-(2,3,6-trifluorophenoxy)ethoxy]phenyl}-1,2,5,6-tetrahydropyridine-3-carboxylic acid cyclopropyl-(2,3-dichlorobenzyl)amide,
4-{4-[2-(2,6-dichloro-4-fluorophenoxy)ethoxy]phenyl}-1,2,5,6-tetrahydro-pyridine-3-carboxylic acid (2-chloro-3-trifluoromethylbenzyl)cyclopropylamide,
4-{4-[2-(2,4,6-trifluorophenoxy)ethoxyphenyl}-1,2,5,6-tetrahydropyridine-3-carboxylic acid (2-chloro-3-trifluoromethylbenzyl)cyclopropylamide,
4-{4-[2-(2,6-difluoro-3-methylphenoxy)ethoxy]phenyl}-1,2,5,6-tetrahydropyridine-3-carboxylic acid cyclopropyl-(2,3-dichlorobenzyl)amide,
4-{4-[2-(4-chloro-2-methoxyphenoxy)ethoxy]phenyl}-1,2,5,6-tetrahydro-pyridine-3-carboxylic acid cyclopropyl-(2,3-dichlorobenzyl)amide,
4-{4-[2-(2,6-dichloro-4-methylphenoxy)ethoxy]phenyl}-1,2,5,6-tetrahydro-pyridine-3-carboxylic acid (6-chlorobenzo[1,3]dioxol-5-ylmethyl)-cyclopropylamide,
4-{4-[2-(2,6-dichloro-4-methylphenoxy)ethoxyphenyl}-1,2,5,6-tetrahydro-pyridine-3-carboxylic acid (2-bromobenzyl)cyclopropylamide,
4-{4-[2-(2,6-dichloro-4-methylphenoxy)ethoxy]phenyl}-1,2,5,6-tetrahydro-pyridine-3-carboxylic acid cyclopropyl-(3-methylbenzyl)amide,
4-{4-[2-(2,6-dichloro-4-methylphenoxy)ethoxy]phenyl}-1,2,5,6-tetrahydro-pyridine-3-carboxylic acid cyclopropyl-(3,5-dimethoxybenzyl)amide,
4-{4-[2-(2,6-dichloro-4-methylphenoxy)ethoxy]phenyl}-1,2,5,6-tetrahydro-pyridine-3-carboxylic acid (3-chlorobenzyl)cyclopropylamide,
4-{4-[2-(2,6-dichloro-4-fluorophenoxy)ethoxy)phenyl}-1,2,5,6-tetrahydro-pyridine-3-carboxylic acid cyclopropyl-(2,3-dimethylbenzyl)amide,
4-{4-[2-(2-chloro-4,5-dimethylphenoxy)ethoxy]phenyl}-1,2,5,6-tetrahydro-pyridine-3-carboxylic acid (2-chloro-3-trifluoromethylbenzyl)cyclopropylamide,
4-{4-[2-(2,6-dichloro-4-methylphenoxy)ethoxy]phenyl}-1,2,5,6-tetrahydro-pyridine-3-carboxylic acid (2-chlorobenzyl)ethylamide,
4-{4-[2-(2,6-dichloro-4-methylphenoxy)ethoxy]phenyl}-1,2,5,6-tetrahydro-pyridine-3-carboxylic acid cyclopropyl-(3-methoxybenzyl)amide,
4-{4-[2-(3-chloro-2,6-difluorophenoxy)ethoxy]phenyl}-1,2,5,6-tetrahydro-pyridine-3-carboxylic acid cyclopropyl-(2,3-dimethylbenzyl)amide,
4-{4-[2-(benzo[1,3]dioxol-5-yloxy)ethoxy]phenyl}-1,2,5,6-tetrahydro-pyridine-3-carboxylic acid (2-chloro-3-trifluoromethylbenzyl)cyclopropylamide,
4-{4-[2-(2,6-dichloro-4-methylphenoxy)ethoxy]phenyl}-1,2,5,6-tetrahydro-pyridine-3-carboxylic acid cyclopropyl-(3-trifluoromethoxybenzyl)amide,
4-{4-[2-(2,6-dichloro-4-fluorophenoxy)ethoxy]phenyl}-1,2,5,6-tetrahydro-pyridine-3-carboxylic acid cyclopropyl-(3,5-difluorobenzyl)amide,
4-{4-[2-(2,6-dichloro-4-methylphenoxy)ethoxy]phenyl}-1,2,5,6-tetrahydro-pyridine-3-carboxylic acid cyclopropyl-(3,4-dimethoxybenzyl)amide,
4-{4-[2-(2,4,6-trifluorophenoxy)ethoxy]phenyl}-1,2,5,6-tetrahydropyridine-3-carboxylic acid cyclopropyl-(2,3-dimethylbenzyl)amide,
4-{4-[2-(2-bromo-5-fluorophenoxy)ethoxy]phenyl}-1,2,5,6-tetrahydro-pyridine-3-carboxylic acid cyclopropyl-(2,3-dimethylbenzyl)amide,
4-{4-[2-(2,3,6-trifluorophenoxy)ethoxy]phenyl}-1,2,5,6-tetrahydropyridine-3-carboxylic acid cyclopropyl-(2,3-dimethylbenzyl)amide,
4-{4-[2-(3-chloro-2,6-difluorophenoxy)ethoxy]phenyl}-1,2,5,6-tetrahydro-pyridine-3-carboxylic acid cyclopropyl-(2,3-dichlorobenzyl)amide,
4-{4-[2-(2,4,6-trifluorophenoxy)ethoxy]phenyl}-1,2,5,6-tetrahydropyridine-3-carboxylic acid (2-bromobenzyl)cyclopropylamide,
4-{4-[2-(2-bromo-5-fluorophenoxy)ethoxy]phenyl}-1,2,5,6-tetrahydro-pyridine-3-carboxylic acid (2-chloro-3-trifluoromethylbenzyl)cyclopropylamide,
4-{4-[2-(benzo[1,3]dioxol-5-yloxy)ethoxy]phenyl}-1,2,5,6-tetrahydro-pyridine-3-carboxylic acid cyclopropyl-(2,3-dichlorobenzyl)amide formate salt,
4-{4-[2-(4-chloro-2-methoxyphenoxy)ethoxy]phenyl}-1,2,5,6-tetrahydro-pyridine-3-carboxylic acid (2-chloro-3-trifluoromethylbenzyl)cyclopropylamide,
4-{4-[2-(4-chloro-2-methoxyphenoxy)ethoxy]phenyl}-1,2,5,6-tetrahydro-pyridine-3-carboxylic acid cyclopropyl-(2,3-dimethylbenzyl)amide,
4-{4-[2-(2,6-dichloro-4-methylphenoxy)ethoxy]phenyl}-1,2,5,6-tetrahydro-pyridine-3-carboxylic acid cyclopropyl-(2-fluoro-5-methoxybenzyl)amide,
4-{4-[2-(2,5-dichlorophenoxy)ethoxy]phenyl}-1,2,5,6-tetrahydropyridine-3-carboxylic acid cyclopropyl-(2,3-dichlorobenzyl)amide,
4-{4-[2-(2-chloro-4,5-dimethylphenoxy)ethoxy]phenyl}-1,2,5,6-tetrahydropyridine-3-carboxylic acid cyclopropyl-(2,3-dimethylbenzyl)amide,
4-{4-[2-(2-chloro-4-trifluoromethylphenoxy)ethoxy]phenyl}-1,2,5,6-tetrahydro-pyridine-3-carboxylic acid (2-chloro-3-trifluoromethylbenzyl)-cyclopropylamide,
4-{4-[2-(2,6-difluoro-3-methylphenoxy)ethoxy]phenyl}-1,2,5,6-tetrahydro-pyridine-3-carboxylic acid (2-chloro-3-trifluoromethylbenzyl)cyclopropylamide,
4-{4-[2-(2-chloro-4-trifluoromethylphenoxy)ethoxy]phenyl}-1,2,5,6-tetrahydropyridine-3-carboxylic acid cyclopropyl-(2,3-dimethylbenzyl)amide,
4-{4-[2-(2,5-dichlorophenoxy)ethoxy]phenyl}-1,2,5,6-tetrahydropyridine-3-carboxylic acid (2-chloro-3-trifluoromethylbenzyl)cyclopropylamide,
4-{4-[2-(2,5-dichlorophenoxy)ethoxy]phenyl}-1,2,5,6-tetrahydropyridine-3-carboxylic acid cyclopropyl-(2,3-dimethylbenzyl)amide,
4-{4-[2-(2-chloro-4-trifluoromethylphenoxy)ethoxy]phenyl}-1,2,5,6-tetrahydropyridine-3-carboxylic acid cyclopropyl-(2,3-dichlorobenzyl)amide,
4-{4-[2-(2-bromo-5-fluorophenoxy)ethoxy]phenyl}-1,2,5,6-tetrahydropyridine-3-carboxylic acid cyclopropyl-(2,3-dichlorobenzyl)amide,
4-{4-[2-(2,3-Dichlorophenoxy)ethoxy]phenyl}-1,2,5,6-tetrahydropyridine-3-carboxylic acid cyclopropyl-(2,3-dichlorobenzyl)amide,
4-{4-[2-(2-chloro-5-fluorophenoxy)ethoxy]phenyl}-1,2,5,6-tetrahydropyridine-3-carboxylic acid cyclopropyl-(2,3-dichlorobenzyl)amide,
4-{4-[2-(2,5-dichlorophenoxy)ethoxy]phenyl}-1,2,5,6-tetrahydropyridine-3-carboxylic acid (2-bromobenzyl)cyclopropylamide,
4-{4-[2-(4-chloro-2-methylphenoxy)ethoxy]phenyl}-1,2,5,6-tetrahydropyridine-3-carboxylic acid cyclopropyl-(2,3-dichlorobenzyl)amide,
4-{4-[2-(2,6-dichloro-4-fluorophenoxy)ethoxy]phenyl}-1,2,5,6-tetrahydro-pyridine-3-carboxylic acid cyclopropyl-(3-trifluoromethoxybenzyl)amide,
4-{4-[2-(2,4,6-trifluorophenoxy)ethoxy]phenyl}-1,2,5,6-tetrahydropyridine-3-carboxylic acid cyclopropyl-(2-fluoro-5-methoxybenzyl)amide,
4-{4-[2-(2,6-dichloro-4-fluorophenoxy)ethoxy]phenyl}-1,2,5,6-tetrahydro-pyridine-3-carboxylic acid cyclopropyl-(3,5-dimethoxybenzyl)amide,
4-{4-[2-(2-chloro-4,5-dimethylphenoxy)ethoxy]phenyl-1,2,5,6-tetrahydro-pyridine-3-carboxylic acid cyclopropyl-(2-fluoro-5-methoxybenzyl)amide,
4-{4-[2-(5-chloro-2-methoxyphenoxy)ethoxy]phenyl}-1,2,5,6-tetrahydro-pyridine-3-carboxylic acid (2-bromobenzyl)cyclopropylamide,
4-{4-[2-(2,3,6-trifluorophenoxy)ethoxy]phenyl}-1,2,5,6-tetrahydropyridine-3-carboxylic acid (2-bromobenzyl)cyclopropylamide,
4-{4-[2-(2-chloro-4,5-dimethylphenoxy)ethoxy]phenyl}-1,2,5,6-tetrahydro-pyridine-3-carboxylic acid cyclopropyl-(3,5-dimethoxybenzyl)amide,
4-{4-[2-(2-chloro-4,5-dimethylphenoxy)ethoxy]phenyl}-1,2,5,6-tetrahydro-pyridine-3-carboxylic acid (2-bromobenzyl)cyclopropylamide,
4-{4-[2-(2,6-dichloro4-fluorophenoxy)ethoxy]phenyl}-1,2,5,6-tetrahydro-pyridine-3-carboxylic acid (3-chlorobenzyl)cyclopropylamide,
4-{4-[2-(2,6-difluoro-3-methylphenoxy)ethoxy]phenyl)}-1,2,5,6-tetrahydro-pyridine-3-carboxylic acid cyclopropyl-(2,3-dimethylbenzyl)amide,
4-{4-[2-(2,6-dichloro-4-fluorophenoxy)ethoxy]phenyl}-1,2,5,6-tetrahydro-pyridine-3-carboxylic acid cyclopropyl-(2-fluoro-5-methoxybenzyl)amide,
4-{4-[2-(2,6-difluoro-3-methylphenoxy)ethoxy]phenyl}-1,2,5,6-tetrahydro-pyridine-3-carboxylic acid (2-bromobenzyl)cyclopropylamide,
4-{4-[2-(2-fluorophenoxy)ethoxy]phenyl}-1,2,5,6-tetrahydropyridine-3-carboxylic acid (2-chloro-3-trifluoromethylbenzyl)cyclopropylamide,
4-{4-[2-(2,4,6-trifluorophenoxy)ethoxy]phenyl}-1,2,5,6-tetrahydropyridine-3-carboxylic acid (6-chlorobenzo[1,3]dioxol-5-ylmethyl)cyclopropylamide,
4-{4-[2-(3-chloro-2,6-difluorophenoxy)ethoxy]phenyl}-1,2,5,6-tetrahydro-pyridine-3-carboxylic acid (6-chlorobenzo[1,3]dioxol-5-ylmethyl)-cyclopropylamide,
4-{4-[2-(2,6-dichloro-4-methylphenoxy)ethoxy]phenyl}-1,2,5,6-tetrahydro-pyridine-3-carboxylic acid cyclopropyl-(3,5-difluorobenzyl)amide,
4-{4-[2-(2,4,5-trichlorophenoxy)ethoxy]phenyl}-1,2,5,6-tetrahydropyridine-3-carboxylic acid (2-chloro-3-trifluoromethylbenzyl)cyclopropylamide,
4-{4-[2-(2-chloro-5-fluorophenoxy)ethoxy]phenyl)-1,2,5,6-tetrahydropyridine-3-carboxylic acid (2-chloro-3-trifluoromethylbenzyl)cyclopropylamide,
4-{4-[2-(2,3-dichlorophenoxy)ethoxy]phenyl}-1,2,5,6-tetrahydropyridine-3-carboxylic acid cyclopropyl-(2,3-dimethylbenzyl)amide,
4-{4-[2-(2,6-dichloro-4-fluorophenoxy)ethoxy]phenyl}-1,2,5,6-tetrahydro-pyridine-3-carboxylic acid (2-chlorobenzyl)ethylamide,
4-{4-[2-(2,4,5-trichlorophenoxy)ethoxy]phenyl}-1,2,5,6-tetrahydropyridine-3-carboxylic acid cyclopropyl-(2,3-dimethylbenzyl)amide,
4-{4-[2-(3-chloro-2,6-difluorophenoxy)ethoxy]phenyl}-1,2,5,6-tetrahydro-pyridine-3-carboxylic acid (2-bromobenzyl)cyclopropylamide,
4-{4-[2-(benzo[1,3]dioxol-5-yloxy)ethoxy]phenyl}-1,2,5,6-tetrahydro-pyridine-3-carboxylic acid cyclopropyl-(2,3-dimethylbenzyl)amide,
4-{4-[2-(2-chloro-4-trifluoromethylphenoxy)ethoxy]phenyl}-1,2,5,6-tetrahydro-pyridine-3-carboxylic acid cyclopropyl-(3,5-dimethoxybenzyl)amide,
4-{4-[2-(2,4,6-trifluorophenoxy)ethoxy]phenyl}-1,2,5,6-tetrahydropyridine-3-carboxylic acid cyclopropyl-(3,5-dimethoxybenzyl)amide,
4-{4-[2-(2,4,6-trifluorophenoxy)ethoxy]phenyl}-1,2,5,6-tetrahydropyridine-3-carboxylic acid (2-chlorobenzyl)ethylamide,
4-{4-[2-(2-chloro-4-trifluoromethylphenoxy)ethoxy]phenyl}-1,2,5,6-tetrahydropyridine-3-carboxylic acid cyclopropyl-(2-fluoro-5-methoxy-benzyl)amide,
4-{4-[2-(2,6-dichloro-4-fluorophenoxy)ethoxy]phenyl}-1,2,5,6-tetrahydro-pyridine-3-carboxylic acid (6-chlorobenzo[1,3]dioxol-5-ylmethyl)-cyclopropylamide,
4-{4-[2-(4-chloro-2-methoxyphenoxy)ethoxy]phenyl}-1,2,5,6-tetrahydro-pyridine-3-carboxylic acid cyclopropyl-(3-methoxybenzyl)amide,
4-{4-[2-(2-chloro-4-trifluoromethylphenoxy)ethoxy]phenyl}-1,2,5,6-tetrahydropyridine-3-carboxylic acid (2-bromobenzyl)cyclopropylamide,
4-{4-[2-(2-chloro-4-trifluoromethylphenoxy)ethoxy]phenyl}-1,2,5,6-tetrahydro-pyridine-3-carboxylic acid cyclopropyl-(3-methoxybenzyl)amide,
4-{4-[2-(2,3,6-trifluorophenoxy)ethoxy]phenyl}-1,2,5,6-tetrahydropyridine-3-carboxylic acid (2-chlorobenzyl)ethylamide,
4-{4-[2-(3-chloro-2,6-difluorophenoxy)ethoxy]phenyl}-1,2,5,6-tetrahydro-pyridine-3-carboxylic acid cyclopropyl-(2-fluoro-5-methoxybenzyl)amide,
4-{4-[2-(2-bromo-5-fluorophenoxy)ethoxy]phenyl}-1,2,5,6-tetrahydro-pyridine-3-carboxylic acid cyclopropyl-(2-fluoro-5-methoxybenzyl)amide,
4-{4-[2-(2,5-dichlorophenoxy)ethoxy]phenyl}-1,2,5,6-tetrahydropyridine-3-carboxylic acid cyclopropyl-(3,5-dimethoxybenzyl)amide,
4-{4-[2-(2-chloro-4,5-dimethylphenoxy)ethoxy]phenyl}-1,2,5,6-tetrahydro-pyridine-3-carboxylic acid cyclopropyl-(3-methoxybenzyl)amide,
4-(4-[2-(4-chloro-2-methylphenoxy)ethoxy]phenyl}-1,2,5,6-tetrahydro-pyridine-3-carboxylic acid cyclopropyl-(2,3-dimethylbenzyl)amide,
4-{4-[2-(4-chloro-2-methoxyphenoxy)ethoxy]phenyl}-1,2,5,6-tetrahydro-pyridine-3-carboxylic acid (6-chlorobenzo[1,3]dioxol-5-ylmethyl)-cyclopropylamide,
4-{4-[2-(2-bromo-5-fluorophenoxy)ethoxy]phenyl}-1,2,5,6-tetrahydro-pyridine-3-carboxylic acid (2-bromobenzyl)cyclopropylamide,
4-{4-[2-(3-chloro-2,6-difluorophenoxy)ethoxy]phenyl}-1,2,5,6-tetrahydro-pyridine-3-carboxylic acid cyclopropyl-(3-methoxybenzyl)amide,
4-{4-[2-(2-chloro-4-trifluoromethylphenoxy)ethoxy]phenyl}-1,2,5,6-tetrahydro-pyridine-3-carboxylic acid (6-chlorobenzo[1,3]dioxol-5-ylmethyl)-cyclopropylamide,
4-{4-[2-(2-chloro-4,5-dimethylphenoxy)ethoxy]phenyl}-1,2,5,6-tetrahydro-pyridine-3-carboxylic acid (2-chlorobenzyl)cyclopropylamide,
4-{4-[2-(2,6-dichloro-4-methylphenoxy)ethoxy]phenyl}-1,2,5,6-tetrahydro-pyridine-3-carboxylic acid (2-chlorobenzyl)cyclopropylamide,
4-{4-[2-(2,4,5-trichlorophenoxy)ethoxy]phenyl}-1,2,5,6-tetrahydropyridine-3-carboxylic acid (2-chlorobenzyl)cyclopropylamide,
4-{4-[2-(2-chloro-5-fluorophenoxy)ethoxy]phenyl}-1,2,5,6-tetrahydro-pyridine-3-carboxylic acid (2-chlorobenzyl)cyclopropylamide,
4-{4-[2-(2-chloro-3,6-difluorophenoxy)ethoxy]phenyl}-1,2,5,6-tetrahydro-pyridine-3-carboxylic acid (2-chlorobenzyl)cyclopropylamide,
4-{4-[2-(2-chloro-6-methylphenoxy)ethoxy]phenyl}-1,2,5,6-tetrahydropyridine-3-carboxylic acid (2-chlorobenzyl)cyclopropylamide,
4-{4-[2-(2,3-dichlorophenoxy)ethoxy]phenyl}-1,2,5,6-tetrahydro-pyridine-3-carboxylic acid (2-chlorobenzyl)cyclopropylamide,
4-{4-[2-(2,6-dichloro-4-fluorophenoxy)ethoxy]phenyl}-1,2,5,6-tetrahydro-pyridine-3-carboxylic acid (2-chlorobenzyl)cyclopropylamide,
4-{4-[2-(3-chloro-2,6-difluorophenoxy)ethoxy]phenyl}-1,2,5,6-tetrahydro-pyridine-3-carboxylic acid (2-chlorobenzyl)cyclopropylamide,
4-{4-[2-(2,4,6-trifluorophenoxy)ethoxy]phenyl}-1,2,5,6-tetrahydropyridine-3-carboxylic acid (2-chlorobenzyl)cyclopropylamide,
4-{4-[2-(2,5-dichlorophenoxy)ethoxy]phenyl}-1,2,5,6-tetrahydropyridine-3-carboxylic acid (2-chlorobenzyl)cyclopropylamide, and
4-{4-[2-(2,6-dichlorophenoxy)ethoxy]phenyl)-1,2,5,6-tetrahydropyridine-3-carboxylic acid (2-chlorobenzyl)cyclopropylamide.

The compounds of general formula I and their pharmaceutically acceptable salts may be used as therapeutics e.g. in form of pharmaceutical compositions. They may especially be used in the treatment and/or prophylaxis of cardiovascular and renal diseases. Examples of such diseases are hypertension, coronary diseases, cardiac insufficiency, renal insufficiency, renal and myocardial ischemia, and renal failure. They can also be used to prevent restenosis after balloon or stent angioplasty, to treat erectile dysfunction, glomerulonephritis, renal colic, and glaucoma. Furthermore, they can be used in the therapy and the prophylaxis of diabetic complications, complications of vascular or cardiac surgery or after organ transplantation, complications of cyclosporin treatment, as well as other diseases presently known to be related to the RAS.

In another embodiment, the invention relates to a method for the treatment and/or prophylaxis of diseases which are related to the RAS such as hypertension, coronary diseases, cardiac insufficiency, renal insufficiency, renal and myocardial ischemia, and renal failure, which method comprises administering a compound as defined above to a human being or animal.

The invention farther relates to the use of compounds of general formula I as defined above for the treatment and/or prophylaxis of diseases which are associated with the RAS such as hypertension, coronary diseases, cardiac insufficiency, renal insufficiency, renal and myocardial ischemia, and renal failure.

In addition, the invention relates to the use of compounds as defined above for the preparation of medicaments for the treatment and/or prophylaxis of diseases which are associated with the RAS such as hypertension, coronary diseases, cardiac insufficiency, renal insufficiency, renal and myocardial ischemia, and renal failure. These medicaments may be prepared in a manner known per se.

The compounds of formula I may also be used in combination with one or more other therapeutically useful substances e. g. with other renin inhibitors, with ACE-inhibitors, with angiotensin-receptor antagonists, with diuretics, with calcium channel blockers, with endothelin receptors antagonists or with other drugs beneficial for the prevention or the treatment of cardiovascular events or renal insufficiency.

All forms of prodrugs leading to an active component comprised in general formula I are included in the present invention.

The compounds of general formula I can be manufactured by the methods given below, by the methods given in the examples or by analogous methods.

Preparation of the Precursors:

Precursors are compounds that were prepared as key intermediates and/or building blocks and which were suitable for further transformations in parallel chemistry.

Ideal starting materials are any commercially available 4-oxo-piperidine-3-carboxylic acid ester derivatives, for instance 1-benzyl-4-oxo-piperidine-3-carboxylic acid methyl ester, possibly as a salt. For practical purposes, a transesterification (for instance according to Seebach D., et al., Synthesis, 1982, 138) to another ester derivative A (wherein R^{a} is optionally a lower alkyl, a lower alkenyl, or a benzyl group), thereafter a change in the N-protecting group (PG: all abreviations are outlined at the beginning of the chapter Examples) to a derivative of type B, may be necessary (Scheme 1).

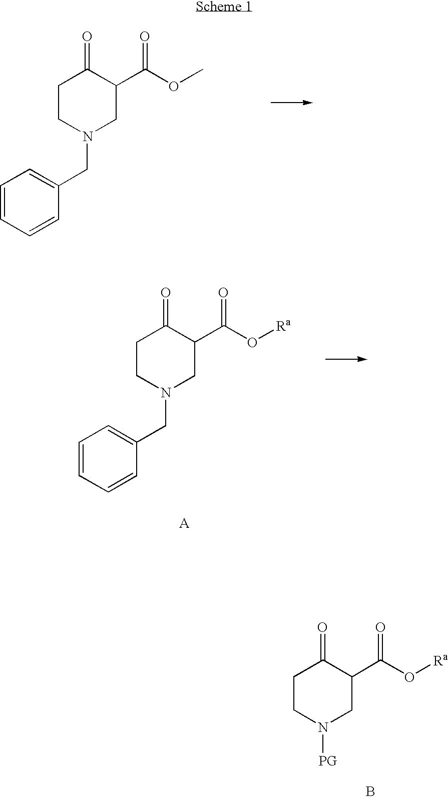

Formation of the vinyl triflate C, followed by a coupling catalysed by a Pd(0) complex may lead to tetrahydropyridine derivatives of type D, wherein R^{b} optionally represents any U-V group as defined in general formula I or a chemical precursor of such a group (Scheme 2).

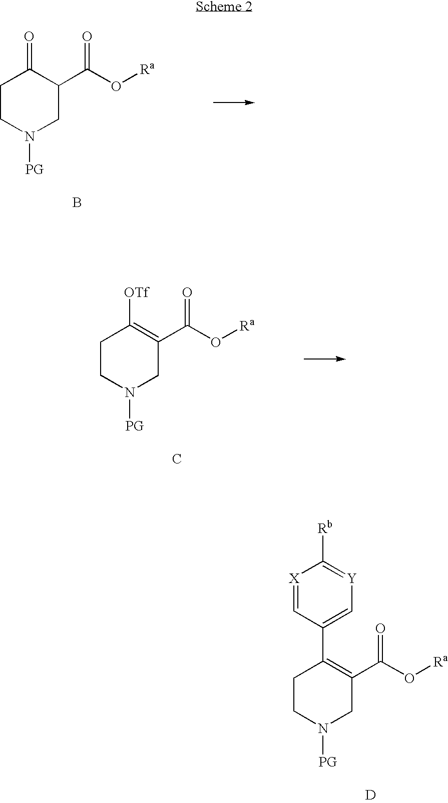

If, for instance, R^{b} is a linker ending with a silanyl ether, compounds of type D are deprotected to compounds of type E, then coupled to a phenol or aromatic alcohol using a Mitsunobu reaction, leading to derivatives of type F wherein V and U have the meaning given in general formula I above (Scheme 3). The ester F is optionally then be cleaved by any suitable method to lead to precursor G.

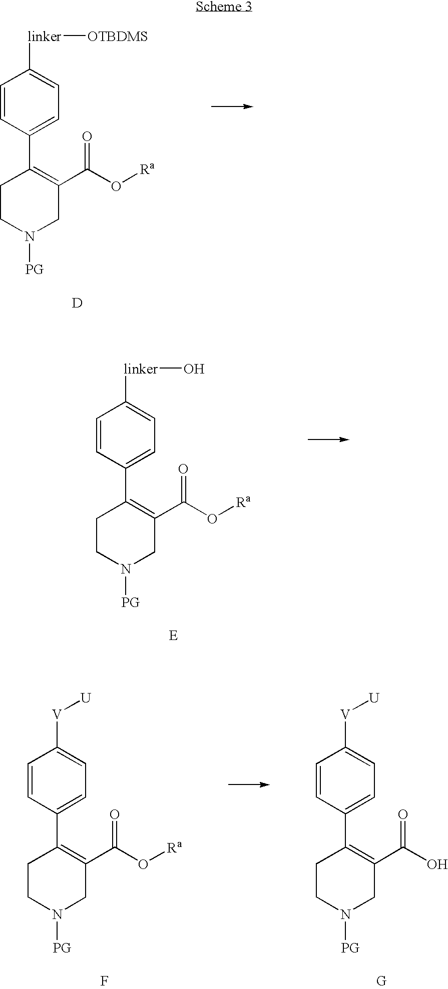

Also, a compound of type D may be reduced with DIBAL to a compound of type M that can be then oxidized to a compound of type N with e.g. the Dess-Martin periodinane (Scheme 4). Aldehyde N may then be transformed to a compound of type O by reductive amination, which can be acylated to a derivative of type Q′ wherein Q and M have the meaning given in general formula I above. On the other hand, compounds of type M can be then acylated following standard procedures to esters or carbamates of type P.

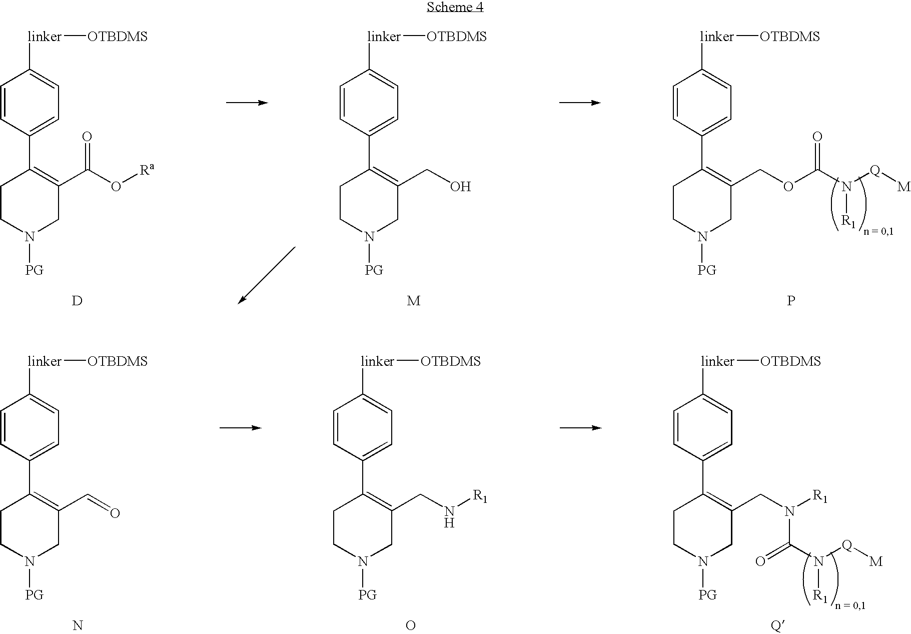

Also, as shown in Scheme 5, a precursor of type T can be prepared in three steps from a compound of type D, by saponification (compound of type R), amide lo coupling (compound of type S) and finally desilylation. 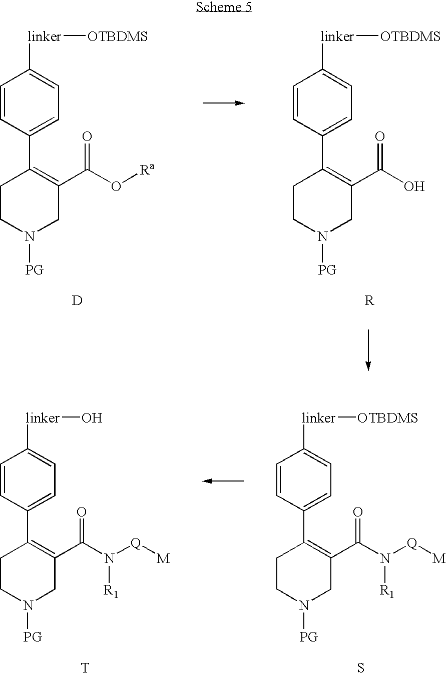
Preparation of Bromoaryl Derivatives

For the coupling of compounds of type C to tetrahydropyridine derivatives of type D, it can be necessary to prepare the bromoaryl components needed as described in Scheme 6. A Mitsunobu coupling (→compounds of type H) or the alkylation of an alcohol with a benzylic chloride (or bromide,→compounds of type J) are often the most convenient methods. Derivative K was prepared in one step from 1-(3-chloropropoxymethyl)-2-methoxybenzene by reaction with 4-bromophenol (Vieira E. et al., Bioorg. Med. Chem. Letters, 1999, 9, 1397). Other methods for the preparation of ethers or thioethers, like a Williamson synthesis, might be used as well (see e.g. March, J, “Advanced Organic Chemistry”, 5^{th} ed., John Wiley and sons, 2001). 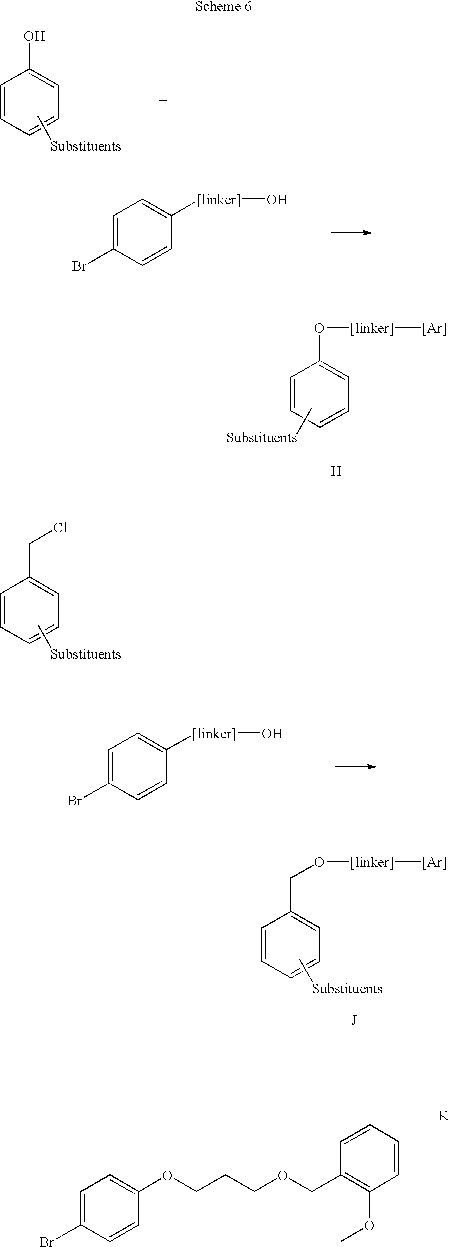
Preparation of the Secondary Amines

It may be necessary to prepare secondary amines as well. This can be done by reductive amination from the corresponding amine and aldehyde, or by amide coupling, from the corresponding amine and carboxylic acid, followed by reduction with LAH or borane. These standard procedures are well-described in the literature.

Preparation of Final Compounds

A compound of type G can be coupled to the amine to yield amides of type L wherein V, U and M have the meaning given in general formula I above. Removal of the N-protecting group (PG) leads to a final compound, wherein V, U, Q and M have the meaning given in general formula I above (Scheme 7).

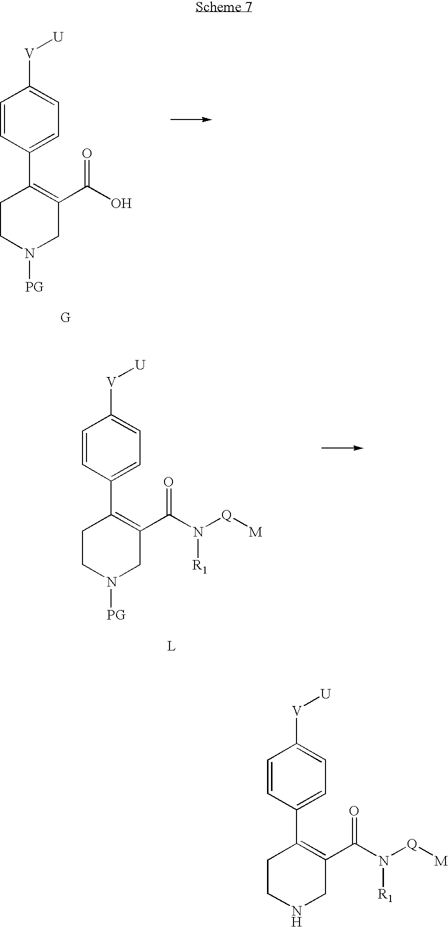

Also, compounds of type P or Q′ (Scheme 4) may be processed further as indicated in Scheme 3, then deprotected as indicated in Scheme 7, to lead to final compounds as defined in general formula I.

From a precursor of type T a final compound can be prepared by a Mitsunobu-type reaction, followed by deprotection (Scheme 8).

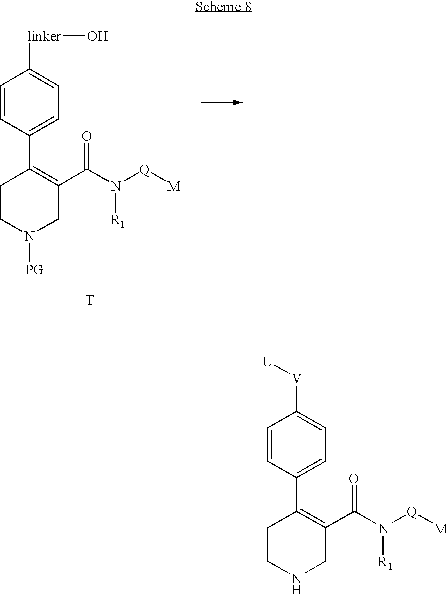

The compounds of formula I and their pharmaceutically acceptable acid addition salts can be used as medicaments, e. g. in the form of pharmaceutical preparations for enteral, parenteral, or topical administration. They can be administered, for example, perorally, e. g. in the form of tablets, coated tablets, dragées, hard and soft gelatine capsules, solutions, emulsions or suspensions, rectally, e. g. in the form of suppositories, parenterally, e. g. in the form of injection solutions or infusion solutions, or topically, e. g. in the form of ointments, creams or oils.

The production of pharmaceutical preparations can be effected in a manner which will be familiar to any person skilled in the art by bringing the described compounds of formula I and their pharmaceutically acceptable acid addition salts, optionally in combination with other therapeutically valuable substances, into a galenical administration form together with suitable, non-toxic, inert, therapeutically compatible solid or liquid carrier materials and, if desired, usual pharmaceutical adjuvants in a manner known per se.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragees and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of compounds of formula I can vary within wide limits depending on the disease to be controlled, the age and the individual condition of the patient and the mode of administration, and will, of course, be fitted to the individual requirements in each particular case. For adult patients a daily dosage of about 1 mg to about 1000 mg, especially about 50 mg to about 500 mg, comes into consideration. For children the dosage has to be adapted to the body weight and age.

The pharmaceutical preparations conveniently contain about 1-500 mg, preferably 5-200 mg of a compound of formula I.

The following examples serve to illustrate the present invention in more detail. They are, however, not intended to limit its scope in any manner.

### EXAMPLES

General Remarks

The following compounds were prepared according to the procedures described for the synthesis of compounds encompassed by the general formula I. All compounds were characterized by ¹H-NMR (300 MHz) and occasionally by ¹³C-NMR (75 MHz) (Varian Oxford, 300 MHz), by LC-MS: A: 2 min <t_{R}<10 min; (Waters Micromass; ZMD-platform with ESI-probe with Alliance 2790 HT; Column: 2×30 mm, Gromsil ODS4, 3 μM, 120A; Gradient: 0-100% acetonitril in water, 6 min, with 0.05% formic acid, flow: 0.45 mL/min; t_{R} given in min.), B: 0.1 min <t_{R}<2 min; (Finnigan AQA with ESI-probe with HP 110 DAD and HP110 binary pump; column: Develosil RP-AQUEOUS, 5 μM, 4.6 mm×50 mm; gradient: 5-95% methanol in water (0.04% TFA), 1 min, 95% methanol in water (0.04% TFA) 0.4 min, 4.5 mL/min.), by TLC (TLC-plates from Merck, Silica gel 60 F₂₅₄). LC-MS- and TLC-data only are given hereby.

Abbreviations

ACE Angiotensin Converting Enzyme
Ang Angiotensin
aq. aqueous
Bn Benzyl
Boc tert-Butyloxycarbonyl
BSA Bovine serum albumine
BuLi n-Butyllithium
conc. Concentrated
DIBAL Diisobutylaluminium hydride
DIPEA Diisopropylethylanline
DMAP 4-N,N-Dimethylaminopyridine
DMF N,N-Dimethylformamide
DMSO Dimethylsulfoxide
EDC.HCl Ethyl-N,N-dimethylaminopropylcarbodiimide hydrochloride
EIA Enzyme immunoassay
eq. equivalent
Et Ethyl
EtOAc Ethyl acetate
FC Flash Chromatography
HOBt Hydroxybenzotriazol
LAH Lithium aluminium hydride
MeOH Methanol
org. organic
PBS Phosphate Buffer Saline
PG protecting group
Ph Phenyl
RAS Renin Angiotensin System
RP18 Reversed phase column, filled with C₁₈ hydrocarbon
rt room temperature
sol. Solution
TBDMS tert-Butyldimethylsilyl
Tf Trifluoromethylsulfonyl
TFA Trifluoroacetic acid
THF Tetrahydrofuran
TLC Thin Layer Chromatography
TMAD N,N,N′,N′-Tetramethylazodicarboxamide
   General Procedures
   General Procedure A for Amide Coupling

A sol. of the desired carboxylic acid (1.00 eq), the desired amine (2.00 eq), EDC.HCl (1.10 eq.), HOBt (cat. amount), DMAP (cat. amount) and DIPEA (2.00 eq.) in CH₂Cl₂ (20 mL/g of acid) was stirred at rt overnight. The reaction mixture was either washed over diatomic earth (Isolute Sorbent Technology, Johnson, C. R., et al., Tetrahedron, 1998, 54, 4097), or washed with aq. 1M HCl, and the org. extracts were evaporated under reduced pressure. The residue was used without further purification.

General Procedure B for the Removal of a Boc-Protecting Group

The starting material was dissolved in CH₂Cl₂ (10 mL/g of starting material) and the sol. was cooled to 0° C. 4M HCl in dioxane (same volume as CH₂Cl₂) was added and the reaction mixture was left for 90 min at rt. The solvents were removed under reduced pressure. Purification of the residue by HPLC led to the desired compound.

Typical Procedure C for Amide Formation from Acid Chlorides

To a sol. of the acid chloride (1 eq.) in CH₂Cl₂ (2.5 mL/mmol) at 0° C. the amine (3 eq.) was added. The mixture was stirred for 3 h while warming up slowly to rt. If necessary, more CH₂Cl₂ was added, then the reaction mixture was washed with aq. sat. NaHCO₃ (1×) and aq. 1M HCl (1×). The extracts were dried over MgSO₄ and the solvents were removed under reduced pressure. The obtained product was used without further purification.

Typical Procedure D for the Reduction of an Amide to an Amine with LAH

To a sol. of the amide (1 eq.) was dissolved in THF (3 mL/mmol) LAH (1M in THF, 3 eq.) was added carefully. The mixture was stirred at rt for 30 min, then heated to 60° C. for 3 h before it was allowed to cool down to rt, then to 0° C. For ×g of LAH initially added, was added ×g of water, then ×g of aq. 15% NaOH, and finally 3×g of water again. The resulting mixture was stirred overnight, filtered, and the precipitate washed with EtOAc. The filtrate was evaporated under reduced pressure and the residue diluted in a small amount of MeOH. The sol. was passed through a pad of SCX silica gel (sulfonic acid). Elution started with MeOH, followed by NH₃/MeOH. The amines eluted with the second second eluent. The solvents were removed under reduced pressure. The isolated amines were either used without further purification or purified by HPLC, depending on the purity.

Typical Procedure E for Reductive Amination

To a solution of aldehyde (1 eq.) in MeOH (0.5 mL/mmol) was added an amine (1.2 eq.). The solution was stirred for 2 h. Sodium borohydride (1.2 eq.) was added portionwise at 0° C. and then stirring was continued, at rt, for 4 h. A solution of NaOH IN was added and the MeOH was evaporated. The mixture was extracted with EtOAc twice and the organic layer was washed with brine, dried over Na₂SO₄ and filtered. The solvent was removed under reduced pressure. The isolated amines were either used without further purification or purified by flash chromatography (EtOAc/heptane: 2/8), depending on the purity.

Preparation of the Secondary Amines

### (2-Chlorobenzyl)cyclopropylamine

Synthesized according to typical procedures C and D from 2-chlorobenzoyl chloride and cyclopropylamine.

### (2-Chlorobenzyl)ethylamine

See Ishihara, Y; et al.; Chem. Pharm. Bull., 1991, 39, 3225.

### Cyclopropyl-(3,5-dimethoxybenzyl)amine

Synthesized according to typical procedure E from 2,5-dimethoxybenzaldehyde and cyclopropylamine.

### Cyclopropyl-(2-fluoro-5-methoxybenzyl)amine

Synthesized according to typical procedure E from 2-fluoro-5-methoxybenzaldehyde and cyclopropylamine.

### Cyclopropyl-(3-methoxybenzyl)amine

Synthesized according to typical procedure E from 3-methoxybenzaldehyde and cyclopropylamine.

### Cyclopropyl-(3,4-dimethoxybenzyl)amine

Synthesized according to typical procedure E from 3,4-dimethoxybenzaldehyde and cyclopropylamine.

### (2-Chloro-3-trifluoromethylbenzyl)cyclopropylamine

Synthesized according to typical procedure E from 2-chloro-3-trifluoromethylbenzaldehyde and cyclopropylamine.

### (6-Chlorobenzo[1,3]dioxol-5-ylmethyl)cyclopropylamine

Synthesized according to typical procedure E from 6-chlorobenzo[1,3]dioxole-5-carbaldehyde and cyclopropylamine.

### (2-Bromobenzyl)cyclopropylamine

Synthesized according to typical procedure E from 2-bromobenzaldehyde and cyclopropylamine.

### Cyclopropyl-(2,3-dimethylbenzyl)amine

Synthesized according to typical procedure E from 2,3-dimethylbenzaldehyde and cyclopropylamine.

### Cyclopropyl-(3,5-difluorobenzyl)amine

Synthesized according to typical procedure E from 3,5-difluorobenzaldehyde and cyclopropylamine.

### (2,3-Dichlorobenzyl)cyclopropylamine

Synthesized according to typical procedure E from 2,3-dichlorobenzaldehyde and cyclopropylamine.

### Cyclopropyl-(3-trifluoromethoxybenzyl)amine

Synthesized according to typical procedure E from 3-trifluoromethoxy-benzaldehyde and cyclopropylamine.

### Cyclopropyl-(3-methylbenzyl)amine

Synthesized according to typical procedure E from 3-methylbenzaldehyde and cyclopropylamine.

### (3-Chlorobenzyl)cyclopropylamine

Synthesized according to typical procedure E from 3-chlorobenzaldehyde and cyclopropylamine.

### Cyclopropyl(2-fluorobenzyl)amine

Synthesized according to typical procedure E from 2-fluorobenzaldehyde and cyclopropylamine.

### Cyclopropyl-(2-methylbenzyl)amine

Synthesized according to typical procedure E from 2-methylbenzaldehyde and cyclopropylamine.

### Cyclopropyl-[2-(4-methoxyphenoxy)ethyl]amine

Synthesized according to typical procedures C and D from (4-methoxyphenoxy)-acetic acid and cyclopropylamine.

### Cyclopropyl-[2-(3-methoxyphenoxy)ethyl]amine

Synthesized according to typical procedures C and D from (3-methoxyphenoxy)-acetic acid and cyclopropylamine.

### Cyclopropyl-(2-m-tolyloxyethyl)amine

Synthesized according to typical procedures C and D from m-tolylacetic acid and cyclopropylamine.

### [2-(2-Chlorophenyl)ethyl]cyclopropylamine

Synthesized according to typical procedures C and D from (2-chlorophenyl)-acetic acid and cyclopropylamine.

### Cyclopropyl-[2-(4-fluorophenyl)ethyl]amine

Synthesized according to typical procedures C and D from (4-fluorophenyl)acetic acid and cyclopropylamine.

### Cyclopropyl-(2-o-tolylethyl)amine

Synthesized according to typical procedures C and D from o-tolylacetic acid and cyclopropylamine.

### Cyclopropyl-(2-p-tolylethyl)amine

Synthesized according to typical procedures C and D from p-tolylacetic acid and cyclopropylamine.

Preparation of the Precursors

### 4-Oxopiperidine-1,3-dicarboxylic acid 1-tert-butyl ester 3-methyl ester (B)

A suspension of 1-benzyl-4-oxopiperidine-3-carboxylic acid methyl ester hydrochloride (5.00 g, 17.6 mmol), triethylamine (2.45 mL, 17.6 mmol) and Boc₂O (4.20 g, 20.0 mmol) in EtOH (30 mL) was purged with N₂. Pd/C (10%, 600 mg) was added and the suspension purged with H₂. The reaction mixture was stirred under an H₂-atmosphere for 24 h and then filtered through Celite. The filtrate was evaporated under reduced pressure. Purification of the residue by FC (EtOAc/heptane 1:4→2:3) yielded the title compound (4.02 g, 89%). R_{f}=0.60 (EtOAc/heptane 1:1). LC-MS: Rₜ=1.09 min, ES+=202.03.

Compounds of Type C

### 1-Benzyl-4-trifluoromethanesulfonyloxy-1,2,5,6-tetrahydropyridine-3-carboxylic acid ethyl ester (C1)

To a suspension of 1-benzyl-4-oxo-piperidine-3-carboxylic acid ethyl ester hydrochloride (1.50 g, 5.04 mmol) in THF (30 mL) NaH (about 60% in oil, 600 mg, about 15 mmol) was added at 0° C. As the suspension turned thick CH₂Cl₂ (20 mL) was added. The ice bath was removed and Tf₂NPh (2.68 g, 7.50 mmol) was added. The mixture was stirred overnight and ice was added. The mixture was washed with aq. 10% Na₂CO₃ (1×) and the org. extracts were dried over MgSO₄ and filtered. The solvents were removed under reduced pressure and purification of the residue by FC (EtOA/heptane 1:9→1:4→2:3) yielded the title compound (2.10 g, almost quantitative yield). R_{f}=0.50 (EtOAc/heptane 1:1). LC-MS: Rₜ=4.65 min, ES+: 394.12.

### 4-Trifluoromethanesulfonyloxy-5,6-dihydro-2H-pyridine-1,3-dicarboxylic acid 1-tert-butyl ester 3-methyl ester (C2)

To a sol. of compound B (4.00 g, 15.6 mmol) in THF (100 mL) at 0° C. was added NaH (suspension in oil, 55-65%, 1.20 g, about 31 mmol). The suspension was stirred for 30 min at 0° C. and Tf₂NPh (8.27 g, 23.1 mmol) was added. The ice bath was removed and the reaction mixture stirred for 3 days at rt. Ice was added and the solvents were removed under reduced pressure. The residue was diluted with EtOAc and washed with aq. 10% Na₂CO₃. The org. extracts were dried over MgSO₄, filtered and the solvent removed under reduced pressure. Purification of the residue by SC (EtOAc/heptane 1:4) yielded the title compound (5.19 g, 86%). LC-MS: Rₜ=1.17, ES+=374.96.

Compounds of Type D

### 1-Benzyl-4-{4-[3-(2-methoxybenzyloxy)propoxy]phenyl}-1,2,5,6-tetrahydro-pyridine-3-carboxylic acid ethyl ester (D1)

To a sol. of 4-bromo-1-[3-(2-methoxybenzyloxy)propoxy]benzene (2.81 g, 8.01 mmol) in THF (50 mL) at −78° C. n-BuLi (1.5M in hexane, 5.60 mL, 8.41 mmol) was added. After 30 min ZnCl₂ (1M in THF, 9.00 mL, 9.00 mmol) was added and the mixture was allowed to warm up to rt. Vinyl triflate C1 (2.10 g, 5.34 mmol) and Pd(PPh₃)₄ (154 mg, 0.134 mmol) were added and the mixture stirred at rt for 4.5 h. Ice was added, the mixture was diluted with EtOAc and washed with aq. 1M NaOH (1×). The org. extracts were dried over MgSO₄, filtered, and the solvents were removed under reduced pressure. Purification of the residue by FC (EtOAc/heptane 1:9→1:4→2:3→3:2) led to the title compound (2.25 g, 82%). R_{f}=0.32 (EtOAc/heptane 1:1). LC-MS: Rₜ=4.05 min, ES+=516.23.

### 4-{4-[3-(tert-Butyldimethylsilanyloxy)propyl]phenyl}-5,6-dihydro-2H-pyridine-1,3-dicarboxylic acid 1-tert-butyl ester 3-methyl ester (D2)

To a sol. of [3-(4-bromophenyl)propoxy]-tert-butyldimethylsilane (Kiesewetter D. O., Tetrahedron Asymmetry, 1993, 4, 2183; 6.19 g, 19.7 mmol) in THF (100 mL) at −78° C. was added n-BuLi (1.5M in hexane, 14.0 mL, 21.0 mmol). The sol. was stirred at −78° C. for 30 min and ZnCl₂ (1M in THF, 22.3 mL, 22.3 mmol) was added. The resulting sol. was allowed to warm to rt and compound C2 (5.10 g, 13.1 mmol) and Pd(PPh₃)₄ (300 mg, 0.26 mmol) were added. After 20 min at rt ice was added to the reaction mixture. The solvents were removed under reduced pressure and the residue diluted with EtOAc. This mixture was washed with aq. 1M NaOH. The org. extracts were dried over MgSO₄, filtered and the solvents removed under reduced pressure. Purification of the residue by FC (EtOAc/heptane 1:9) led to the title compound (5.77 g, 90%). LC-MS: Rₜ=7.27 min, ES+=512.54.

### 4-{4-[2-(tert-Butyldimethylsilanyloxy)ethoxy]phenyl}-5,6-dihydro-2H-pyridine-1,3-dicarboxylic acid 1-tert-butyl ester 3-methyl ester (D3)

As described for compound D2 but from [2-(4-bromo-phenoxy)ethoxy]-tert-butyldimethylsilane (Morita, C.; et al.al.; Heterocycles, 2000, 52, 1163; 49.5 g, 149 mmol), BuLi (1.6M in hexane, 94 mL, 150 mmol), ZnCl₂ (1M in THF, 200 mL, 200 mmol), compound C2 (37.0 g, 95 mmol), Pd(PPh₃)₄ (2.75 g, 2.38 mmol) and THF (750 mL). Purification by FC yielded the title compound (36.6 g, 78%). LC-MS: Rₜ=1.20 min, ES+=492.34.

Compounds of Type E

### 4-[4-(3-Hydroxypropyl)phenyl]-5,6-dihydro-2H-pyridine-1,3-dicarboxylic acid 1-tert-butyl ester 3-methyl ester (E1)

TBAF (1.90 g, 6.00 mmol) was added to a sol. of compound D2 (1.95 g, 4.00 mmol) in THF (40 mL). The reaction mixture was stirred for 6 h at rt and diluted with EtOAc. The resulting mixture was washed with water and brine. The org. extracts were dried over MgSO₄, filtered and the solvents removed under reduced pressure. Purification of the residue by FC (EtOAc/heptane 2:3) yielded the title compound (1.27 g, 84%). LC-MS: Rₜ=1.06, ES+=376.18.

### 4-[4-(2-Hydroxyethoxy)phenyl]-5,6-dihydro-2H-pyridine-1,3-dicarboxylic acid 1-tert-butyl ester-3-methyl ester (E2)

As described for compound E1 but from compound D3 (5.63 g, 11.4 mmol), TBAF (5.41 g, 17.1 mmol) and THF (115 mL). Purification by FC yielded the title compound (3.46 g, 80%). LC-MS: Rₜ=1.01; ES+=378.22.

Compounds of Type F

### 4-{4-[3-(2-Bromo-5-fluorophenoxy)propyl]phenyl}-5,6dihydro-2H-pyridine-1,3-dicarboxylic acid 1-tert-butyl ester 3-methyl ester (F1)

A sol. of compound E1 (750 mg, 2.00 mmol), 2-bromo-5-fluorophenol (0.334 mL, 3.00 mmol), azodicarboxyl dipiperidide (757 mg, 3.00 mmol), tri-n-butylphosphine (0.987 mL, 4.00 mmol) and DIPEA ( 0.035 mL, 0.20 mmoL) in toluene (20 mL) was stirred for 1 h at rt, then for 2 h at 60° C. The reaction mixture was allowed to cool to rt, was diluted with EtOAc and washed with water. The org. extracts were dried over MgSO₄, filtered and the solvents were removed under reduced pressure. Purification of the residue by FC (EtOAc/heptane 1:4→3:7) led to the title compound (898 mg, 82%). LC-MS: Rₜ=6.43 min, ES+=570.00.

### 4-{4-[3-(2-Chlorophenoxy)propyl]phenyl}-5,6-dihydro-2H-pyridine-1,3-dicarboxylic acid 1-tert-butyl ester 3-methyl ester (F2)

A sol. of compound E1 (375 mg, 1.00 mmol), 2-chlorophenol (0.153 mL, 1.50 mmol), azodicarboxyl dipiperidide (378 mg, 1.50 mmol), tri-n-butylphosphine (0.493 mL, 2.00 mmol) and DIPEA ( 0.018 mL, 0.10 mmoL) in toluene (10 mL) was stirred for 1 h at rt, then for 2 h at 60° C. The reaction mixture was allowed to cool to rt, was diluted with EtOAc and washed with water. The org. extracts were dried over MgSO₄, filtered and the solvents were removed under reduced pressure. Purification of the residue by FC (EtOAc/heptane 1:4→3:7) led to the title compound (374 mg, 77%). LC-MS: Rₜ=1.39 min, ES+=486.13.

### 4-{4-[3-(2,5-Difluorophenoxy)propyl]phenyl}-5,6-dihydro-2H-pyridine-1,3-dicarboxylic acid 1-tert-butyl ester 3-methyl ester (F3)

A sol. of compound E1 (375 mg, 1.00 mmol), 2,5-difluorophenol (195 mg, 1.50 mmol), azodicarboxyl dipiperidide (378 mg, 1.50 mmol), tri-n-butylphosphine (0.493 mL, 2.00 mmol) and DIPEA ( 0.018 mL, 0.10 mmoL) in toluene (10 mL) was stirred for 1 h at rt, then for 2 h at 60° C. The reaction mixture was allowed to cool to rt, was diluted with EtOAc and washed with water. The org. extracts were dried over MgSO₄, filtered and the solvents were removed under reduced pressure. Purification of the residue by FC (EtOAc/heptane 1:4→3:7) led to the title compound (378 mg, 77%). LC-MS: Rt=1.35 min, ES+=488.16.

### 4-{4-[3-(2,3,6-Trifluorophenoxy)propyl]phenyl}-5,6-dihydro-2H-pyridine-1,3-dicarboxylic acid 1-tert-butyl ester 3-methyl ester (F4)

Prepared as described for compound F1but from compound E1 (4.7 g, 12.5 mmol), 2,3,6-trifluorophenol (3.7 g, 25.0 mmol), azodicarboxyl dipiperidide (6.32 g, 34.2 mmol), tributylphosphine (85%, 9.3 mL, 37.6 mmol) and toluene (100 mL). Purification of the residue by FC yielded the title compound (5.23 g, 83%).

### 4-{4-[2-(2,3,5-Trimethylphenoxy)ethyl]phenyl}-5,6-dihydro-2H-pyridine-1,3-dicarboxylic acid 1-tert-butyl ester 3-methyl ester (F5)

As described for compound D2 but from compound H1 (3.07 g, 9.63 mmol), BuLi (1.6M in hexane, 6.9 mL, 10.3 mmol), ZnCl₂ (1M in THF, 10.9 mL, 10.9 mmol), compound C2 (2.50 g, 6.42 mmol), Pd(PPh₃)₄ (148 mg, 0.128 mmol) and THF (50 mL). Purification by FC yielded the title compound (1.77 g, 57%).

### 4-{4-[2-(2-Chloro-4,5-dimethylphenoxy)ethoxy]phenyl}-5,6-dihydro-2H-pyridine-1,3-dicarboxylic acid 1-tert-butyl ester 3-methyl ester (F6)

Prepared as described for compound F1but from compound E2 (1.69 g, 4.4 mmol), 2-chloro-4,5-dimethylphenol (1.05 g, 6.6 mmol), azodicarboxyl dipiperidide (1.67 g, 6.6 mmol), tributylphosphine (2.2 mL, 8.8 mmol) and toluene (45 mL). Purification of the residue by FC yielded the title compound (1.73 g, 76%). LC-MS: Rt=1.38; ES+: 516.24.

Compounds of Type G

### 4-{1-[3-(2-Bromo-5-fluorophenoxy)propyl]phenyl}-5,6-dihydro-2H-pyridine-1,3-dicarboxylic acid 1-tert-butyl ester (G1)

To a sol. of compound F1(742 mg, 1.30 mmol) in EtOH (13 mL) was added aq. 1M NaOH (13 mL). The resulting mixture was stirred for 35 min at 80° C., then allowed to cool to rt. Aq. 1M HCl (13 mL) was added and the resulting mixture was extracted with EtOAc (3×). The combined org. extracts were dried over MgSO₄, filtered and the solvents were removed under reduced pressure. Purification of the residue by FC (EtOAc/heptane 2:3) led to the title compound (418 mg, 60%). LC-MS: Rₜ=1.32 min, ES+=534.04.

### 4-{4-[3-(2-Chlorophenoxy)propyl]phenyl}-5,6-dihydro-2H-pyridine-1,3-dicarboxylic acid 1-tert-butyl ester (G2)

To a sol. of compound F2 (374 mg, 0.77 mmol) in EtOH (8 mL) was added aq. 1M NaOH (7.7 mL). The resulting mixture was stirred for 35 min at 80° C., then allowed to cool to rt. Aq. 1M HCl (7.7 mL) was added and the resulting mixture was extracted with EtOAc (3×). The combined org. extracts were dried over MgSO₄, filtered and the solvents were removed under reduced pressure. Purification of the residue by FC (EtOAc/heptane 2:3) led to the title compound (218 mg, 60%). LC-MS: Rₜ=1.29 min, ES+=472.15.

### 4-{4-[3-(2,5-Difluorophenoxy)propyl]phenyl}-5,6dihydro-2H-pyridine-1,3-dicarboxylic acid 1-tert-butyl ester (G3)

To a sol. of compound F3 (378 mg, 0.77 mmol) in EtOH (8 mL) was added aq. 1M NaOH (7.7 mL). The resulting mixture was stirred for 35 min at 80° C., then allowed to cool to rt. Aq. 1M HCl (7.7 mL) was added and the resulting mixture was extracted with EtOAc (3×). The combined org. extracts were dried over MgSO₄, filtered and the solvents were removed under reduced pressure. Purification of the residue by FC (EtOAc/heptane 2:3) led to the title compound (220 mg, 60%). LC-MS: Rₜ=1.25 min, ES+=474.17.

### 4-{1-[3-(2,3,6-Trifluorophenoxy)propyl]phenyl}-5,6-dihydro-2H-pyridine-1,3-dicarboxylic acid 1-tert-butyl ester (G4)

As described for compound G1, but from compound F4 (5.23 g, 10.3 mmol), aq. NaOH (1M, 90 mL) and EtOH (90 mL). The title product was used further without chromatographic purification (4.55 g, 89%).

### 4-{4-[2-(2,3,5-Trimethylphenoxy)ethyl]phenyl}-5,6-dihydro-2H-pyridine-1,3-dicarboxylic acid 1-tert-butyl ester (G5)

As described for compound G1, but from compound F5 (2.17 g, 4.53 mmol), aq. NaOH (1M, 30 mL) and EtOH (30 mL). The title product was used further without chromatographic purification (1.86 g, 89%).

### 4-{4-[2-(2-Chloro-4,5-dimethylphenoxy)ethoxy]phenyl}-5,6-dihydro-2H-pyridine-1,3-dicarboxylic acid 1-tert-butyl ester (G6)

As described for compound G1, but from compound F6 (1.73 g, 3.3 mmol), aq. NaOH (1M, 33 mL) and EtOH (33 mL). The title product was used further without chromatographic purification. LC-MS: Rₜ=1.10; ES+: 502.31.

### 2-(4-Bromophenyl)eth-1-yl 2,3,5-trimethylphenyl ether (H1)

A mixture of 2-(4-bromophenyl)ethanol (20.0 mL, 143 mmol), 2,3,5-trimethylphenol (31.1 g, 229 mmol), azodicarboxylic dipiperidide (72.1 g, 286 mmol) and tributylphosphine (88 mL; 357 mmol) in toluene (2.00 L) was heated to reflux for 2 h. The mixture was allowed to cool to rt. The mixture was filtered, washed with toluene and the solvents were partially removed under reduced pressure. The residue was diluted with Et₂O and washed with aq. 1M NaOH (2×). The org. extracts were dried over MgSO₄, filtered, and the solvents were removed under reduced pressure. Purification of the residue by FC (petroleum ether→Et₂O/petroleum ether 1:3) yielded the title compound (33.1 g, 73%). LC-MS: Rₜ=6.95.

### 1-Bromo-4-[3-(2-methoxybenzyloxy)prop-1-yloxy]benzene (K)

4-Bromophenol (4.32 g, 25.0 mmol) and 1-(3-chloro-propoxymethyl)-2-methoxy-benzene (Vieira E., et al., Bioorg. Med. Chem. Letters, 1999, 9, 1397) (4.88 g, 22.7 mmoL) were dissolved in DMF (150 mL). NaI (1.50 g, 0.10 mmol) and Cs₂CO₃ (16.3 g, 50.0 mmol) were added. The mixture was heated to 80° C. and stirred for 6 h before it was allowed to cool to rt. After dilution with EtOAc (600 mL) the mixture was washed with water (1×), aq. 1M NaOH (1×), and aq. 1M HCl (1×). The org. extracts were dried over MgSO₄ and filtered. The solvents were removed under reduced pressure. Purification of the residue by FC (Et2O/petroleum ether 1:9→1:4) yielded the title compound (5.66 g, 71%). R_{f}=0.60 (Et₂O/heptane 1:1). ¹H-NMR (CDCl₃): 7.38-7.34 (m, 3 H); 7.26 (t, J=8.7 Hz, 1 H); 6.94 (t, J=8.7 Hz, 1 H); 6.86 (d, J=8.2 Hz, 1 H); 6.78 (d, J=9.0 Hz, 2 H); 4.57 (s, 2 H); 4.07 (t, J=6.3 Hz, 2 H); 3.81 (s, 3 H); 3.70 (t, J=6.3 Hz, 2 H), 2.10 (quint., J=6.3 Hz, 2 H).

### 1-Benzyl-4-{4-[3-(2-methoxybenzyloxy)propoxy]phenyl}-I,2,5,6-tetrahydro-pyridine-3-carboxylic acid [1-(2-chlorophenyl)ethyl]methylamide (L1)

To a suspension of tetrahydropyridine D1 (2.25 g, 4.26 mmol) in EtOH (50 mL) NaOH (1M in water, 30 mL) was added. After 4 h the mixture was warmed up to 60° C. and stirred for 5 h. The reaction mixture was allowed to cool to rt and the pH was adjusted to 7 with aq. 1M HCl. The solvents were removed under reduced pressure and the residue was dried at high vacuum. The dried residue was triturated with EtOH and filtered (3×), the combined filtrates were evaporated under reduced pressure, and the residue was dried at high vacuum. The residue was diluted in CHCl₃ (20 mL), and [2-(2-chlorophenyl)ethyl]methylamine (Jaques B.; Wallace R. G., Tetrahedron, 1977, 33, 581, 1.48 g, 8.72 mmol), DMAP (cat. amount), HOBt (cat. amount) and EDC.HCl (836 mg, 4.36 mmol) were added. After 4 h at rt the mixture was diluted with CH₂Cl₂ and washed with aq. 10% Na₂CO₃ (1×). The org. extracts were dried over MgSO₄, filtered, and the solvents were removed under reduced pressure. Purification of the residue by FC (EtOAc/heptane 1:4→1:3→2:3→3:2 EtOAc) gave the title compound (0.48 g, 17%). R_{f}=0.13 (EtOAcheptane 1:1). LC-MS: Rₜ=4.24 min, ES+=639.33.

### 4-{4-[1-(tert-Butyldimethylsilanyloxy)ethoxy]phenyl}-5,6-dihydro-2H-pyridine-1,3-dicarboxylic acid 1-tert-butyl ester (R)

A sol. of compound D3 (17.6 g) in MeOH (400 ml) and IN NaOH-soln. (250 ml) was heated at 110° C. for 1.5 h. The mixture was allowed to cool to rt and aq. 1M HCl was added to reach pH 4, and was extracted with EtOAc (2×1 50 ml). The org. extracts were dried over MgSO₄, filtered, and the solvents were removed under reduced pressure. A sol. this crude material (14 g), imidazol (9.75 g) and TBDMSCl (13.49 g) in DMF (80 ml) was stirred at room temperature for 1 h. Aq. sat. NH₄Cl (100 ml) was added and the mixture was extracted with heptane (3×100 ml). ).The org. extracts were dried over MgSO₄, filtered, and the solvents were removed under reduced pressure. A sol. of this crude product, and K₂CO₃ (2.5 g) in MeOH (50 ml) and water (50 ml) was stirred at room temperature for 1 h. Aq. sat. NH₄Cl (100 ml) was added and the mixture was extracted with Et₂O (3×50 ml). ).The org. extracts were dried over MgSO₄, filtered, and the solvents were removed under reduced pressure. The crude title product (17.2 g, quant. yield) was used in the next step without purification. LC-MS: Rₜ=1.12; ES+:478.38.

Compounds of Type S

### 4-{4-[2-(tert-Butyldimethylsilanyloxy)ethoxy]phenyl}-5-[(2-chloro3-trifluoromethylbenzyl)cyclopropylcarbamoyl]-3,6-dihydro-2H-pyridin-1-carboxylic acid tert-butyl ester (S1)

A sol. of compound R (2.62 g, 5.5 mmol), (2-chloro-3-trifluoromethylbenzyl)-cyclopropylamine (2.74 g, 11.0 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL) was stirred overnight. The mixture was washed with aq. 1M HCl (3×) and aq. sat. NaHCO₃ (1×). The org. extracts were dried over MgSO₄, filtered, and the solvents were removed under reduced pressure. Purification of the residue by FC (EtOAc/heptane 1:9→1:4→1:3) yielded the title compound (2.95 g, 75%). R_{f}=0.55 (EtOAc/heptane 1:1). LC-MS: Rₜ=7.68.

### 4-{4-[2-(tert-Butyldimethylsilanyloxy)ethoxy]phenyl}-5-[cyclopropyl-(3,5-difluorobenzyl)carbamoyl]-3,6-dihydro-2H-pyridine-1-carboxylic acid tert-butyl ester (S2)

As described for compound S1, but from compound R (2.62 g, 5.5 mmol), cyclopropyl-(3,5-difluorobenzyl)amine (2.01 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.83 g, 79%). LC-MS: Rₜ=1.20; ES+: 643.23.

### 4-{4-[2-(tert-Butyldimethylsilanyloxy)ethoxy]phenyl}-5-[cyclopropyl-(2,3-dichlorobenzyl)carbamoyl]-3,6-dihydro-2H-pyridin-1-carboxylic acid tert-butyl ester (S3)

As described for compound S1, but from compound R (2.62 g, 5.5 mmol), cyclopropyl-(2,3-dichlorobenzyl)amine (2.38 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.02 g, 53%). LC-MS: Rₜ=1.20; ES+: 675.15.

### 5-[(2-Bromobenzyl)cyclopropylcarbamoyl]-4-{4-[2-(tert-butyldimethyl-silanyloxy)ethoxy]phenyl}-3,6-dihydro-2Hi-pyridine-1-carboxylic acid tert-butyl ester (S4)

As described for compound S1, but from compound R (2.62 g, 5.5 mmol), (2-bromobenzyl)cyclopropylamine (2.49 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.02 g, 53%). LC-MS: Rₜ=1.26; ES+: 687.41.

### 4-{4-[2-(tert-Butyldimethylsilanyloxy)ethoxy]phenyl}-5-[cyclopropyl-(2,3-dimethylbenzyl)carbamoyl]-3,6-dihydro-2H-pyridine-1-carboxylic acid tert-butyl ester (S5)

As described for compound S1, but from compound R (2.62 g, 5.5 mmol), cyclopropyl-(2,3-dimethylbenzyl-amine (1.93 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.25 g, 64%). LC-MS: Rₜ=1.26; ES+: 635.53.

### 4-{4-[2-(tert-Butyldimethylsilanyloxy)ethoxy]phenyl}-5-[cyclopropyl-3-trifluoromethoxybenzyl)carbamoyl]-3,6-dihydro-2H-pyridine-1-carboxylic acid tert-butyl ester (S6)

As described for compound S1, but from compound R (2.62 g, 5.5 mmol), cyclopropyl-(3-trifluoromethoxybenzyl)amine (2.54 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.51 g, 66%). LC-MS: Rₜ=1.26; ES+: 691.48.

### 4-{4-[2-(tert-Butyldimethylsilanyloxy)ethoxy]phenyl}-5-[cyclopropyl-(3-methylbenzyl)carbamoyl]-3,6-dihydro-2H-pyridine-1-carboxylic acid tert-butyl ester (S7)

As described for compound S1, but from compound R (2.62 g, 5.5 mmol), cyclopropyl-(3-methylbenzyl)amine (1.77 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.14 g, 62%). LC-MS: Rₜ=1.25; ES+: 621.54.

### 4-{4-[2-(tert-Butyldimethylsilanyloxy)ethoxy]phenyl}-5[(3-chlorobenzyl)-cyclopropylcarbamoyl]-3,6-dihydro-2H-pyridine-1-carboxylic acid tert-butyl ester (S8)

As described for compound S1, but from compound R (2.62 g, 5.5 mmol), (3-chlorobenzyl)cyclopropylamine (1.99 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.44 g, 69%). LC-MS: Rₜ=1.26; ES+: 641.44.

### 4-{4-[2-(tert-Butyldimethylsilanyloxy)ethoxy]phenyl}-5-[(2-chlorobenzyl)-ethylcarbamoyl]-3,6-dihydro-2H-pyridine-1-carboxylic acid tert-butyl ester (S9)

As described for compound S1, but from compound R (2.62 g, 5.5 mmol), (2-chlorobenzyl)ethylamine (1.87 g, 11 mmol), DMAP (132 mg, 1.12 mmol), DIPEA (3.67 mL, 22.0 mmol), HOBt (817 mg, 6.05 mmol) and EDC.HCl (1.58 g, 8.25 mmol) in CH₂Cl₂ (70 mL). Purification by FC yielded the title compound (2.31 g, 67%). LC-MS: Rₜ=1.25; ES+: 629.45.

### 4-{4-[2-(tert-Butyldimethylsilanyloxy)ethoxy]phenyl}-5-[cyclopropyl-(2-fluoro-5-methoxybenzyl)carbamoyl]-3,6-dihydro-2H-pyridine-1-carboxylic acid tert-butyl ester (S10)

As described for compound S1, but from compound R (2.59 g, 5.42 mmol), cyclopropyl-(2-fluoro-5-methoxybenzyl)amine (2.12 g, 10.8 mmol), DMAP (132 mg, 1.12 mmol), DIPEA (3.70 mL, 21.7 mmol), HOBt (732 mg, 5.42 mmol) and EDC.HCl (1.56 g, 8.13 mmol) in CH₂Cl₂ (50 mL). Purification by FC yielded the title compound (2.21 g, 62%).

### 4-{4-[2-(tert-Butyldimethylsilanyloxy)ethoxy]phenyl})-[(6-chlorobenzo[1,3-dioxol-5-ylmethyl)cyclopropylcarbamoyl]-3,6-dihydro-2H-pyridine-1-carboxylic acid tert-butyl ester (S11)

As described for compound S1, but from compound R (2.41 g, 5.05 mmol), (6-chlorobenzo[1,3]dioxol-5-ylmethyl)cyclopropylamine (2.28 g, 10.1 mmol), DMAP (123 mg, 1.01 mmol), DIPEA (3.50 mL, 20.2 mmol), HOBt (682 mg, 5.05 mmol) and EDC.HCl (1.45 g, 7.58 mmol) in CH₂Cl₂ (50 mL). Purification by FC yielded the title compound (1.97 g, 57%).

### 4-{4-[2-(tert-Butyldimethylsilanyloxy)ethoxy]phenyl}-5-[cyclopropyl-3,5-dimethoxybenzyl)carbamoyl]-3,6-dihydro-2H-pyridine-1-carboxylic acid tert-butyl ester (S12)

As described for compound S1, but from compound R (2.80 g, 5.86 mmol), cyclopropyl-(3,5-dimethoxybenzyl)amine (2.43 g, 11.7 mmol), DMAP (143 mg, 1.17 mmol), DIPEA (3.00 mL, 17.6 mmol), HOBt (792 mg, 5.86 mmol) and EDC.HCl (1.68 g, 8.79 mmol) in CH₂Cl₂ (50 mL). Purification by FC yielded the title compound (2.97 g, 76%). LC-MS: Rₜ=1.23; ES+=667.1.

### 4-{4-[2-(tert-Butyldimethylsilanyloxy)ethoxy]phenyl}-5-[cyclopropyl-(3-methoxybenzyl)carbamoyl]-3,6-dihydro-2H-pyridine-1-carboxylic acid tert-butyl ester (S13)

As described for compound S1, but from compound R (2.80 g, 5.86 mmol), cyclopropyl-(3-methoxybenzyl)amine (2.08 g, 11.7 mmol), DMAP (143 mg, 1.17 mmol), DIPEA (3.00 mL, 17.6 mmol), HOBt (792 mg, 5.86 mmol) and EDC.HCl (1.68 g, 8.79 mmol) in CH₂Cl₂ (50 mL). Puritication by FC yielded the title compound (2.68 g, 72%). LC-MS: Rₜ=1.23; ES+=637.3.

### 4-{4-[2-(tert-Butyldimethylsilanyloxy)ethoxy]phenyl}-5-[cyclopropyl-(3,4-dimethoxybenzyl)carbamoyl]-3,6-dihydro-2H-pyridine-1-carboxylic acid tert-butyl ester (S14)

As described for compound S1, but from compound R (2.48 g, 5.19 mmol), cyclopropyl-(3,4-dimethoxybenzyl)amine (2.15 g, 10.4 mmol), DMAP (127 mg, 1.04 mmol), DIPEA (3.60 mL, 20.8 mmol), HOBt (700 mg, 5.19 mmol) and EDC.HCl (1.49 g, 7.79 mmol) in CH₂Cl₂ (50 mL). Purification by FC yielded the title compound (2.92 g, 84%). LC-MS: Rₜ=1.23; ES+=637.3.

### 4-{4-[2-(tert-Butyldimethylsilanyloxy)ethoxy]phenyl}-5-[(2-chlorobenzyl)-cyclopropylcarbamoyl]-3,6-dihydro-2H-pyridine-1-carboxylic acid tert-butyl ester (S15)

As described for compound S1, but from compound R (3.82 g, 8.00 mmol), (2-chlorobenzyl)cyclopropylamine (4.36 g, 24.0 mmol), DMAP (195 mg, 1.60 mmol), DIPEA (5.50 mL, 32.0 mmol), HOBt (1.08 g, 8.00 mmol) and EDC.HCl (2.30 g, 12.0 mmol) in CH₂Cl₂ (70 mL). Purification by FC yielded the title compound (3.10 g, 60%). LC-MS: Rₜ=1.26; ES+=641.4.

Compounds of Type T

### 5-[(2-Chloro-3-trifluoromethylbenzyl)cyclopropylcarbamoyl]-4-[4-(2-hydroxyethoxy)phenyl]-3,6-dihydro-2H-pyridine-1-carboxylic acid tert-butyl ester (T1)

A sol. of compound S1 (2.95 g, 4.16 mmol) and TBAF (1M in THF, 6.24 mL, 6.24 mmol) in THF (15 mL) was stirred at rt for 90 min. The mixture was diluted with EtOAc and washed with brine (1×), water (1×) and brine again (1×). The org. extracts were dried over MgSO₄, filtered, and the solvents were removed under reduced pressure. Purification of the residue by FC (EtOAc/heptane 1:4→2:3→3:2→4:1) yielded the title compound (1.56 g, 63%). R_{f}=0.10 (EtOAc/heptane 1:1) were collected. LC-MS: Rₜ=5.63; ES+=595.37.

### 5-[Cyclopropyl-(3,5-difluorobenzyl)carbamoyl]-4-[4-(2-hydroxyethoxy)-phenyl]-3,6-dihydro-2H-pyridine-1-carboxylic acid tert-butyl ester (T2)

As described for compound T1, but from compound S2 (2.83 g, 4.40 mmol), TBAF (1M in THF, 6.60 mL, 6.60 mmol) and THF (15 mL). Purification by FC yielded the title compound (0.95 g, 41%). LC-MS: Rₜ=5.16; ES+=529.48.

### 5-[Cyclopropyl-(2,3-dichlorobenzyl)carbamoyl]-4-[4-(2-hydroxyethoxy)-phenyl]-3,6-dihydro-2H-pyridine-1-carboxylic acid tert-butyl ester (T3)

As described for compound T1, but from compound S3 (2.47 g, 3.66 mmol), TBAF (1M in THF, 5.48 mL, 5.48 mmol) and THF (15 mL). Purification by FC yielded the title compound (1.43 g, 70%). LC-MS: Rₜ=5.52; ES+=561.31.

### 5-1(2-Bromobenzyl)cyclopropylcarbamoyl]-4-[4-(2-hydroxyethoxy)phenyl]-3,6-dihydro-2H-pyridine-1-carboxylic acid tert-butyl ester (T4)

As described for compound T1, but from compound S4 (2.02 g, 2.95 mmol), TBAF (1M in THF, 4.42 mL, 4.42 mmol) and THF (15 mL). Purification by FC yielded the title compound (1.40 g, 83%). LC-MS: Rₜ=5.22; ES+=571.32.

### 5-[Cyclopropyl-(2,3-dimethylbenzyl)carbamoyl]-4-[4-(2-hydroxyethoxy)-phenyl]-3,6-dihydro-2H-pyridine-1-carboxylic acid tert-butyl ester (T5)

As described for compound T1, but from compound S5 (2.25 g, 3.54 mmol), TBAF (1M in THF, 5.32 mL, 5.32 mmol) and THF (15 mL). Purification by FC yielded the title compound (1.74 g, 94%). LC-MS: Rₜ=5.32; ES+=521.68.

### 5-[Cyclopropyl-(3-trifluoromethoxybenzyl)carbamoyl]-4-[4-(2-hydroxy-ethoxy)phenyl]-3,6-dihydro-2H-pyridine-1-carboxylic acid tert-butyl ester (T6)

As described for compound T1, but from compound S6 (2.51 g, 3.63 mmol), TBAF (1M in THF, 5.45 mL, 5.45 mmol) and THF (15 mL). Purification by FC yielded the title compound (1.94 g, 93%). LC-MS: Rₜ=1.04; ES+=577.32.

### 5-[Cyclopropyl-(3-methylbenzyl)carbamoyl]-4-[4-(2-hydroxyethoxy)phenyl]-3,6-dihydro-2H-pyridine-1-carboxylic acid tert-butyl ester (T7)

As described for compound T1, but from compound S7 (2.14 g, 3.45 mmol), TBAF (1M in THF, 5.20 mL, 5.20 mmol) and THF (15 mL). Purification by FC yielded the title compound (1.66 g, 95%). LC-MS: Rₜ=5.19; ES+=507.58.

### 5-[(3-Chlorobenzyl)cyclopropylcarbamoyl]4-[4-(2-hydroxyethoxy)phenyl]-3,6-dihydro-2H-pyridine-1-carboxylic acid tert-butyl ester (T8)

As described for compound T1, but from compound S8 (2.44 g, 3.80 mmol), TBAF (1M in THF, 5.70 mL, 5.70 mmol) and THF (15 mL). Purification by FC yielded the title compound (1.71 g, 85%). LC-MS: Rₜ=5.25; ES+=527.37.

### 5-[(2-Chlorobenzyl)ethylcarbamoyl]-4-[4-(2-hydroxyethoxy)phenyl]-3,6-dihydro-2H-pyridine-1-carboxylic acid tert-butyl ester (T9)

As described for compound T1, but from compound S9 (2.31 g, 3.67 mmol), TBAF (1M in THF, 5.50 mL, 5.50 mmol) and THF (15 mL). Purification by FC yielded the title compound (1.40 g, 74%). LC-MS: Rₜ=5.19; ES+=559.06.

### 5-[Cyclopropyl-(2-fluoro-5-methoxybenzyl)carbamoyl]-4-[4-(2-hydroxy-ethoxy)phenyl]-3,6-dihydro-2H-pyridine-1-carboxylic acid tert-butyl ester (T10)

As described for compound T1, but from compound S10 (1.97 g, 2.87 mmol), TBAF (1M in THF, 5.75 mL, 5.75 mmol) and THF (20 mL). Purification by FC yielded the title compound (1.50 g, 97%). LC-MS: Rₜ=5.02; ES+=541.46.

### 5-[(6-Chlorobenzo[1,3]dioxol-5-ylmethyl)cyclopropylcarbamoyl]-4-[4-(2-hydroxyethoxy)phenyl]-3,6-dihydro-2H-pyridine-1-carboxylic acid tert-butyl ester (T11)

As described for compound T1, but from compound S11 (2.20 g, 3.37 mmol), TBAF (1M in THF, 6.75 mL, 6.75 mmol) and THF (25 mL). Purification by FC yielded the title compound (1.58 g, 82%). LC-MS: Rₜ=5.28; ES+=571.34.

### 5-[Cyclopropyl-(3,5-dimethoxybenzyl)carbamoyl]-4-[4-(2-hydroxyethoxy)-phenyl]-3,6-dihydro-2H-pyridine-1-carboxylic acid tert-butyl ester (T12)

As described for compound T1, but from compound S12 (2.97 g, 4.45 mmol), TBAF (1M in THF, 8.90 mL, 8.90 mmol) and THF (30 mL). Purification by FC yielded the title compound (2.14 g, 87%). LC-MS: Rₜ=0.99; ES+=553.2.

### 5-[Cyclopropyl-(3-methoxybenzyl)carbamoyl]-4-[4-(2-hydroxyethoxy)-phenyl]-3,6-dihydro-2H-pyridine-1-carboxylic acid tert-butyl ester (T13)

As described for compound T1, but from compound S13 (2.68 g, 4.21 mmol), TBAF (1M in THF, 8.40 mL, 8.40 mmol) and THF (30 mL). Purification by FC yielded the title compound (2.03 g, 92%). LC-MS: Rₜ=0.97; ES+=523.2.

### 5-[Cyclopropyl-(3,4-dimethoxybenzyl)carbamoyl]-4-[4-(2-hydroxyethoxy)-phenyl]-3,6-dihydro-2H-pyridine-1-carboxylic acid tert-butyl ester (T14)

As described for compound T1, but from compound S14 (2.92 g, 4.38 mmol), TBAF (1M in THF, 8.80 mL, 8.80 mmol) and THF (30 mL). Purification by FC yielded the title compound (2.02 g, 83%). LC-MS: Rₜ=0.96; ES+=553.21.

### 5-[(2-Chlorobenzyl)cyclopropylcarbamoyl]-4-[4-(2-hydroxyethoxy)phenyl]-3,6-dihydro-2H-pyridine-1-carboxylic acid tert-butyl ester (T15)

As described for compound T1, but from compound S15 (3.10 g, 4.84 mmol), TBAF (1M in THF, 10.3 mL, 10.3 mmol) and THF (40 mL). Purification by FC yielded the title compound (2.35 g, 92%). LC-MS: Rₜ=1.02; ES+=527.14.

Preparation of the Final Compounds

### Example 1

### 4-{4-[3-(2-Methoxybenzyloxy)propoxy]phenyl}-1,2,5,6-tetrahydropyridine-3-carboxylic acid [2-(2-chlorophenyl)ethyl]methylamide trifluoroacetate salt

To a sol. of tetrahydropyridine L1 (410 mg, 0.641 mmol) in CH₂ClCH₂Cl (10 mL) at rt ClCO₂CHClCH₃ (0.350 mL, 3.21 mmol) was added. The sol. was stirred at rt for 1 h, then heated to reflux. After 5 h another portion of ClCO₂CHClCH₃ (0.350 mL, 3.21 mmol) was added. After 1 h the solvents were removed under reduced pressure, and the residue was diluted with MeOH (5 mL) and water (5 mL). The mixture was stirred overnight and the solvents were partially removed under reduced pressure. The residue was diluted with EtOAc and the mixture was washed with aq. 1M NaOH (1×). The org. extracts were dried over MgSO₄, filtered, and the solvents were removed under reduced pressure. Purification of the residue by HPLC (H₂O, MeOH, TFA) yielded the title compound (31 mg). LC-MS: Rₜ=3.98 min, ES+=593.13.

### Example 2

### 4-{4-[3-(2-Bromo-5-fluorophenoxy)propyl]phenyl}-1,2,5,6-tetrahydropyridine-3-carboxylic acid [2-(2-chlorophenyl)ethyl]methylamide trifluoroacetate salt

According to the general procedures A and B, starting from compound G1 and [2-(2-chlorophenyl)ethyl]methylamine (Jaques, B.; Wallace, R. G., Tetrahedron, 1977, 33, 581). LC-MS: Rₜ=1.04 min, ES+=586.96.

### Example 3

### 4-{4-[3-(2-Bromo-5-fluorophenoxy)propyl]phenyl}-1,2,5,6-tetrahydropyridine-3-carboxylic acid 2-phenethylmethylamide trifluoroacetate salt

According to the general procedures A and B, starting from compound G1 and methylphenethylamine. LC-MS: Rₜ=1.01 min, ES+=553.01.

### Example 4

### 4-{4-[3-(2-Bromo-5-fluorophenoxy)propyl]phenyl}-1,2,5,6-tetrahydropyridine-3-carboxylic acid (2-chlorobenzyl)methylamide trifluoroacetate salt

According to the general procedures A and B, starting from compound G1 and (2-chlorobenzyl)methylamine (Holzgrabe, U.; Arch. Pharm (Weinheim, Ger.), 1987, 320, 647). LC-MS: Rₜ=1.03 min, ES+=572.95.

### Example 5

### 4-{4-[3-(2-Bromo-5-fluorophenoxy)propyl]phenyl}-1,2,5,6-tetrahydropyridine-3-carboxylic acid (2-chlorobenzyl)cyclopropylamide trifluoroacetate salt

According to the general procedures A and B, starting from compound G1 and (2-chlorobenzyl)cyclopropylamine. LC-MS: Rₜ=1.07 min, ES+=598.98.

### Example 6

### 4-{4-[3-(2-Chlorophenoxy)propyl]phenyl}-1,2,5,6-tetrahydropyridine-3-carboxylic acid [2-(2-chlorophenyl)ethyl]methylamide formate salt

According to the general procedures A and B, starting from compound G2 and [2-(2-chlorophenyl)ethyl]methylamine (Jaques, B.; Wallace, R. G., Tetrahedron, 1977, 33, 581). LC-MS: Rₜ=0.99 min, ES+=523.02.

### Example 7

### 4-{4-[3-(2-Chlorophenoxy)propyl]phenyl}-1,2,5,6-tetrahydropyridine-3-carboxylic acid 2-phenethylmethylamide formate salt

According to the general procedures A and B, starting from compound G2 and methylphenethylamine. LC-MS: Rₜ=0.96 min, ES+=489.07.

### Example 8

### 4-{4-[3-(2-Chlorophenoxy)propyl]phenyl}-1,2,5,6-tetrahydropyridine-3-carboxylic acid (2-chlorobenzyl)methylamide formate salt

According to the general procedures A and B, starting from compound G2 and (2-chlorobenzyl)methylamine (Holzgrabe, U.; Arch. Pharm (Weinheim, Ger.), 1987, 320, 647). LC-MS: Rₜ=0.98 min, ES+=509.01.

### Example 9

### 4-{4-[3-(2-Chlorophenoxy)propyl]phenyl}-1,2,5,6-tetrahydropyridine-3-carboxylic acid (2-chlorobenzyl)cyclopropylamide formate salt

According to the general procedures A and B, starting from compound G2 and (2-chlorobenzyl)cyclopropylamine. LC-MS: Rₜ=1.02 min, ES+=535.06.

### Example 10

### 4-{4-[3-(2,5-Difluorophenoxy)propyl]phenyl}-1,2,5,6-tetrahydropyridine-3-carboxylic acid [2-(2-chlorophenyl)ethyl]methylamide formate salt

According to the general procedures A and B, starting from compound G3 and [2-(2-chlorophenyl)ethyl]methylamine (Jaques, B.; Wallace, R. G., Tetrahedron, 1977, 33, 581). LC-MS: Rₜ=0.97 min, ES+=525.03.

### Example 11

### 4-{4-[3-(2,5-Difluorophenoxy)propyl]phenyl}-1,2,5,6-tetrahydropyridine-3-carboxylic acid 2-phenethylmethylamide formate salt

According to the general procedures A and B, starting from compound G3 and methylphenethylamine. LC-MS: Rₜ=0.94 min, ES+=491.10.

### Example 12

### 4-{4-[3-(2,5-Difluorophenoxy)propyl]phenyl)}-1,2,5,6-tetrahydropyridine-3-carboxylic acid (2-chlorobenzyl)methylamide formate salt

According to the general procedures A and B, starting from compound G3 and (2-chlorobenzyl)methylamine (Holzgrabe, U.; Arch. Pharm. (Weinheim, Ger.), 1987, 320, 647). LC-MS: Rₜ=0.96 min, ES+=511.01.

### Example 13

### 4-{4-[3-(2,5-Difluorophenoxy)propyl]phenyl}-1,2,5,6-tetrahydropyridine-3-carboxylic acid (2-chlorobenzyl)cyclopropylamide formate salt

According to the general procedures A and B, starting from compound G3 and (2-chlorobenzyl)cyclopropylamine. LC-MS: Rₜ=1.00 min, ES+=537.03.

### Example 14

### 4-{4-[3-(2-Bromo-5-fluorophenoxy)propyl]phenyl}-1,2,5,6-tetrahydro-pyridine-3-carboxylic acid (2-chlorobenzyl)cyclopropylamide trifluoroacetate salt

According to the general procedures A and B, starting from compound G1 and (2-chlorobenzyl)cyclopropylamine. LC-MS: Rₜ=1.07 min, ES+=598.98.

### Example 15

### 4-{4-[3-(2,3,6-Trifluorophenoxy)propyl]phenyl}-1,2,5,6tetrahydropyridine-3-carboxylic acid (2-chlorobenzyl)cyclopropylamide trifluoroacetate salt

According to the general procedures A and B, starting from compound G4 and (2-chlorobenzyl)cyclopropylamine. LC-MS: Rₜ=1.03 min, ES+=555.17.

### Example 16

### 4-{4-[3-(2,3,6-Trifluorophenoxy)propyl]phenyl)}-1,2,5,6-tetrahydropyridine-3-carboxylic acid (2-chlorobenzyl)ethylamide trifluoroacetate salt

According to the general procedures A and B, starting from compound G4 and (2-chlorobenzyl)ethylamine. LC-MS: Rₜ=1.01 min, ES+=543.16.

### Example 17

### 4-{4-[3-(2,3,6-Trifluorophenoxy)propyl]phenyl}-1,2,5,6-tetrahydropyridine-3-carboxylic acid cyclopropyl-(2-fluorobenzyl)amide trifluoroacetate salt

According to the general procedures A and B, starting from compound G4 and cyclopropyl-(2-fluorobenzyl)amine. LC-MS: Rₜ=1.01 min, ES+=539.14.

### Example 18

### 4-{4-[3-(2,3,6Trifluorophenoxy)propyl]phenyl}-1,2,5,6-tetrahydropyridine-3-carboxylic acid cyclopropyl-(3-trifluoromethoxybenzyl)amide trifluoroacetate salt

According to the general procedures A and B, starting from compound G4 and cyclopropyl-(3-trifluoromethoxybenzyl)amine. LC-MS: Rₜ=1.04 min, ES+=589.14.

### Example 19

### 4-{4-[3-(2,3,6-Trifluorophenoxy)propyl]phenyl}-1,2,5,6-tetrahydropyridine-3-carboxylic acid cyclopropyl-(2-methylbenzyl)amide trifluoroacetate salt

According to the general procedures A and B, starting from compound G4 and cyclopropyl-(2-methylbenzyl)amine. LC-MS: Rₜ=1.03 min, ES+=535.17.

### Example 20

### 4-{4-[3-(2,3,6-Trifluorophenoxy)propyl]phenyl}-1,2,5,6-tetrahydropyridine-3-carboxylic acid cyclopropyl-[2-(4-methoxyphenoxy)ethyl]amide trifluoroacetate salt

According to the general procedures A and B, starting from compound G4 and cyclopropyl-[2-(4-methoxyphenoxy)ethyl]amine. LC-MS: Rₜ=1.00 min, ES+=581.33.

### Example 21

### 4-{4-[3-(2,3,6-Trifluorophenoxy)propyl]phenyl}-1,2,5,6-tetrahydropyridine-3-carboxylic acid cyclopropyl-[2-(3-methoxyphenoxy)ethyl]amide trifluoroacetate salt

According to the general procedures A and B, starting from compound G4 and cyclopropyl-[2-(3-methoxyphenoxy)ethyl]amine. LC-MS: Rₜ=1.02 min, ES+=581.34.

### Example 22

### 4-{4-[3-(2,3,6-Trifluorophenoxy)propyl]phenyl}-1,2,5,6-tetrahydropyridine-3-carboxylic acid cyclopropyl-(2-m-tolyloxyethyl)amide trifluoroacetate salt

According to the general procedures A and B, starting from compound G4 and cyclopropyl-(2-m-tolyloxyethyl)amine. LC-MS: Rₜ=1.05 min, ES+=565.31.

### Example 23

### 4-{4-[3-(2,3,6-Trifluorophenoxy)propyl]phenyl}-1,2,5,6-tetrahydropyridine-3-carboxylic acid [2-(2-chlorophenyl)ethyl]cyclopropylamide trifluoroacetate salt

According to the general procedures A and B, starting from compound G4 and [2-(2-chlorophenyl)ethyl]cyclopropylamine. LC-MS: Rₜ=0.93 min, ES+=569.41.

### Example 24

### 4-{4-[3-(2,3,6-Trifluorophenoxy)propyl]phenyl}-1,2,5,6-tetrahydropyridine-3-carboxylic acid cyclopropyl-[2-(4-fluorophenyl)ethyl]amide trifluoroacetate salt

According to the general procedures A and B, starting from compound G4 and cyclopropyl-[2-(4-fluorophenyl)ethyl]amine. LC-MS: Rₜ=0.92 min, ES+=553.51.

### Example 25

### 4-{4-[3-(2,3,6-Trifluorophenoxy)propyl]phenyl}-1,2,5,6-tetrahydropyridine-3-carboxylic acid cyclopropyl-(2-o-tolylethyl)amide trifluoroacetate salt

According to the general procedures A and B, starting from compound G4 and cyclopropyl-(2-o-tolylethyl)amine. LC-MS: Rₜ=0.93 min, ES+=549.47.

### Example 26

### 4-{4-[3-(2,3,6-Trifluorophenoxy)propyl]phenyl}-1,2,5,6-tetrahydropyridine-3-carboxylic acid cyclopropyl-(3,5-dimethoxybenzyl)amide trifluoroacetate salt

According to the general procedures A and B, starting from compound G4 and cyclopropyl-(3,5-dimethoxybenzyl)amine. LC-MS: Rₜ=0.91 min, ES+=581.48.

### Example 27

### 4-{4-[3-(2,3,6-Trifluorophenoxy)propyl]phenyl}-1,2,5,6-tetrahydropyridine-3-carboxylic acid cyclopropyl-(2-p-tolylethyl)amide trifluoroacetate salt

According to the general procedures A and B, starting from compound G4 and cyclopropyl(2-p-tolylethyl)amine. LC-MS: Rₜ=0.93 min, ES+=549.53.

### Example 28

### 4-{4-[2-(2,3,5-Trimethylphenoxy)ethyl]phenyl}-1,2,5,6-tetrahydropyridine-3-carboxylic acid cyclopropyl-(3-trifluoromethylbenzyl)amide trifluoroacetate salt

According to the general procedures A and B, starting from compound G5 and cyclopropyl-(3-trifluoromethylbenzyl)amine LC-MS: Rₜ=0.96 min, ES+=563.46.

### Example 29

### 4-{4-[2-(2,3,5-Trimethylphenoxy)ethyl]phenyl}-1,2,5,6-tetrahydropyridine-3-carboxylic acid cyclopropyl-(2-methylbenzyl)amide trifluoroacetate salt

According to the general procedures A and B, starting from compound G5 and cyclopropyl-(2-methylbenzyl)amine. LC-MS: Rₜ=0.94 min, ES+=509.50.

### Example 30

### 4-{4-[2-(2,3,5-Trimethylphenoxy)ethyl]phenyl}-1,2,5,6-tetrahydropyridine-3-carboxylic acid cyclopropylphenethylamide trifluoroacetate salt

According to the general procedures A and B, starting from compound G5 and cyclopropylphenethylamine. LC-MS: Rₜ=0.94 min, ES+=509.53.

### Example 31

### 4-{4-[3-(2-Bromo-5-fluorophenoxy)propyl]phenyl}-1,2,5,6-tetrahydropyridine-3-carboxylic acid (2-chlorobenzyl)ethylamide trifluoroacetate salt

According to the general procedures A and B, starting from compound G1 and (2-chlorobenzyl)ethylamine. LC-MS: Rₜ=0.92 min, ES+=587.13.

### Example 32

### 4-{4-[3-(2-Bromo-5-fluorophenoxy)propyl]phenyl}-1,2,5,6-tetrahydropyridine-3-carboxylic acid cyclopropyl-(2-methylbenzyl)amide trifluoroacetate salt

According to the general procedures A and B, starting from compound G1 and cyclopropyl-(2-methylbenzyl)amine. LC-MS: Rₜ=0.92 min, ES+=577.20.

### Example 33

### 4-{4-[3-(2-Bromo-5-fluorophenoxy)propyl]phenyl}-1,2,5,6-tetrahydropyridine-3-carboxylic acid cyclopropyl-[2-(4-methoxyphenoxy)ethyl]amide trifluoroacetate salt

According to the general procedures A and B, starting from compound G1 and cyclopropyl-[2-(4-methoxyphenoxy)ethyl]amine. LC-MS: Rₜ=0.91 min, ES+=623.21.

### Example 34

### 4-{4-[3-(2-Bromo-5-fluorophenoxy)propyl]phenyl}-1,2,5,6-tetrahydropyridine-3-carboxylic acid cyclopropylphenethylamide trifluoroacetate salt

According to the general procedures A and B, starting from compound G1 and cyclopropylphenethylamine. LC-MS: Rₜ=0.92 min, ES+=577.19.

### Example 35

### 4-{4-[3-(2-Bromo-5-fluorophenoxy)propyl]phenyl}-1,2,5,6-tetrahydropyridine-3-carboxylic acid cyclopropyl-(2-o-tolylethyl)amide

According to the general procedures A and B, starting from compound G1 and cyclopropyl-(2-o-tolylethyl)amine. LC-MS: Rₜ=0.93 min, ES+=593.19.

### Example 36

### 4-{4-[3-(2-Bromo-5-fluorophenoxy)propyl]phenyl}-1,2,5,6-tetrahydropyridine-3-carboxylic acid cyclopropyl-(3,5-dimethoxybenzyl)amide trifluoroacetate salt

According to the general procedures A and B, starting from compound G1 and cyclopropyl-(3,5-dimethoxybenzyl)amine. LC-MS: Rₜ=0.90 min, ES+=623.38.

### Example 37

### 4-{4-[3-(2-Bromo-5-fluorophenoxy)propyl]phenyl}-1,2,5,6-tetrahydropyridine-3-carboxylic acid cyclopropyl-(2-p-tolylethyl)amide trifluoroacetate salt

According to the general procedures A and B, starting from compound G1 and cyclopropyl-(2-p-tolylethyl)amine. LC-MS: Rₜ=0.95 min, ES+=591.38.

### Example 38

### 4-{4-[2-(2-Chloro-4,5-dimethylphenoxy)ethoxy]phenyl}-1,2,5,6-tetrahydropyridine-3-carboxylic acid (2-chlorobenzyl)cyclopropylamide trifluoroacetate salt

According to the general procedures A and B, starting from compound G6 and (2-chlorobenzyl)cyclopropylamine. LC-MS: Rₜ=0.92 min, ES+=577.20.

### Example 39

### 4-{4-[3-(2,3,6-Trifluorophenoxy)propyl]phenyl}-1,2,5,6-tetrahydropyridine-3-carboxylic acid cyclopropyl-(2-fluoro-5-methoxybenzyl)amide formate salt

According to the general procedures A and B, starting from compound G4 and cyclopropyl-(2-fluoro-5-methoxybenzyl)amine. LC-MS: Rₜ=0.91 min, ES+=569.16.

### Example 40

### 4-{4-[3-(2,3,6-Trifluorophenoxy)propyl]phenyl}-1,2,5,6-tetrahydropyridine-3-carboxylic acid cyclopropyl-(3-methoxybenzyl)amide formate salt

According to the general procedures A and B, starting from compound G4 and cyclopropyl-(3-methoxybenzyl)amine. LC-MS: Rₜ=0.91 min, ES+=551.17.

### Example 41

### 4-{4-[3-(2,3,6-Trifluorophenoxy)propyl]phenyl}-1,2,5,6-tetrahydropyridine-3-carboxylic acid cyclopropyl-(3,4-dimethoxybenzyl)amide formate salt

According to the general procedures A and B, starting from compound G4 and cyclopropyl-(3,4-dimethoxybenzyl)amine. LC-MS: Rₜ=0.88 min, ES+=581.18.

### Example 42

### 4-{4-[3-(2,3,6-Trifluorophenoxy)propyl]phenyl}-1,2,5,6-tetrahydropyridine-3-carboxylic acid (2-chloro-3-trifluoromethylbenzyl)cyclopropylamide formate salt

According to the general procedures A and B, starting from compound G4 and (2-chloro-3-trifluoromethylbenzyl)cyclopropylamine. LC-MS: Rₜ=0.96 min, ES+=623.07.

### Example 43

### 4-{4-[3-(2,3,6-Trifluorophenoxy)propyl]phenyl}-1,2,5,6-tetrahydropyridine-3-carboxylic acid (6-chlorobenzo[1,3]dioxol-5-ylmethyl)cyclopropylamide formate salt

According to the general procedures A and B, starting from compound G4 and (6-chlorobenzo[1,3]dioxol-5-ylmethyl)cyclopropylamine. LC-MS: Rₜ=0.93 min, ES+=599.08.

### Example 44

### 4-{4-[3-(2,3,6-Trifluorophenoxy)propyl]phenyl}-1,2,5,6-tetrahydropyridine-3-carboxylic acid (2-chloro-6-fluorobenzyl)cyclopropylamide formate salt

According to the general procedures A and B, starting from compound G4 and (2-chloro-6-fluorobenzyl)-cyclopropylamine. LC-MS: Rₜ=0.92 min, ES+=573.10.

### Example 45

### 4-{4-[3-(2,3,6-Trifluorophenoxy)propyl]phenyl}-1,2,5,6-tetrahydropyridine-3-carboxylic acid (2-bromobenzyl)cyclopropylamide formate salt

According to the general procedures A and B, starting from compound G4 and (2-bromobenzyl)cyclopropylamine. LC-MS: Rₜ=0.94 min, ES+=601.04.

### Example 46

### 4-{4-[3-(2,3,6Trifluorophenoxy)propyl]phenyl}-1,2,5,6-tetrahydropyridine-3-carboxylic acid cyclopropyl-(2,3-dimethylbenzyl)amide formate salt

According to the general procedures A and B, starting from compound G4 and cyclopropyl-(2,3-dimethylbenzyl)amine. LC-MS: Rₜ=0.94 min, ES+=549.17.

### Example 47

### 4-{4-[3-(2,3,6-Trifluorophenoxy)propyl]phenyl}-1,2,5,6-tetrahydropyridine-3-carboxylic acid cyclopropyl-(3-fluoro-2-methylbenzyl)amide formate salt

According to the general procedures A and B, starting from compound G4 and cyclopropyl-(3-fluoro-2-methylbenzyl)amine. LC-MS: Rₜ=0.93 min, ES+=553.17.

### Example 48

### 4-{4-[3-(2,3,6-Trifluorophenoxy)propyl]phenyl}-1,2,5,6-tetrahydropyridine-3-carboxylic acid cyclopropyl-(2,3-dichlorobenzyl)amide formate salt

According to the general procedures A and B, starting from compound G4 and cyclopropyl-(2,3-dichlorobenzyl)amine. LC-MS: Rₜ=0.95 min, ES+=589.07.

### Example 49

### 4-{4-[3-(2,3,6-Trifluorophenoxy)propyl]phenyl}-1,2,5,6-tetrahydropyridine-3-carboxylic acid cyclopropyl-(3-methylbenzyl)amide formate salt

According to the general procedures A and B, starting from compound G4 and cyclopropyl-(3-methylbenzyl)amine. LC-MS: Rₜ=0.93 min, ES+=535.19.

### Example 50

### 4-{4-[3-(2,3,6-Trifluorophenoxy)propyl]phenyl}-1,2,5,6-tetrahydropyridine-3-carboxylic acid cyclopropyl-(2,3-difluorobenzyl)amide formate salt

According to the general procedures A and B, starting from compound G4 and cyclopropyl-(2,3-difluorobenzyl)amine. LC-MS: Rₜ=0.92 min, ES+=557.15.

### Example 51

### 4-{4-[3-(2,3,6-Trifluorophenoxy)propyl]phenyl}-1,2,5,6-tetrahydropyridine-3-carboxylic acid (3-chlorobenzyl)cyclopropylamide formate salt

According to the general procedures A and B, starting from compound G4 and (3-chlorobenzyl)cyclopropylamine. LC-MS: Rₜ=0.93 min, ES+=555.07.

### Example 52

### 4-{4-[2-(2,6-Dichloro-4-methylphenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,6-dichloro-4-methylphenol. LC-MS: Rₜ=0.97 min, ES+=620.90.

### Example 53

### 4-{4-[2-(2,6-Dichloro-4-methylphenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 (50 mg) and 2,6-dichloro-4-methylphenol. LC-MS: Rₜ=0.98 min, ES+=653.03.

### Example 54

### 4-{4-[2-(2,6-Dichloro-4-methylphenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,6-dichloro-4-methylphenol. LC-MS: Rₜ=0.96 min, ES+=579.12.

### Example 55

### 4-{4-[2-(2,3,6-Trifluorophenoxy)ethoxy]phenyl}-1,2,5,6-tetrahydropyridine-3-carboxylic acid (2-chloro-3-trifluoromethylbenzyl)cyclopropylamide formate salt

According to the general procedures F and B, starting from compound T1 and 2,3,6-trifluorophenol. LC-MS: Rₜ=0.94 min, ES+=625.20.

### Example 56

### 4-{4-[2-(2,6-Dichloro-4-fluorophenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2,6-dichloro-4-fluorophenol. LC-MS: Rₜ=0.94 min, ES+=635.19.

### Example 57

### 4-{4-[2-(2,4,6-Trifluorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,4,6-trifluorophenol. LC-MS: Rₜ=0.93 min, ES+=591.16.

### Example 58

### 4-{4-[2-(2,6-Dichloro-4-fluorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,6-dichloro-4-fluorophenol. LC-MS: Rₜ=0.95 min, ES+=625.21.

### Example 59

### 4-{4-[2-(2-Chloro-4,5-dimethylphenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2-chloro-4,5-dimethylphenol. LC-MS: Rₜ=0.96 min, ES+=601.03.

### Example 60

### 4-{4-[2-(2,3,6-Trifluorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,3,6-trifluorophenol. LC-MS: Rₜ=0.92 min, ES+=591.01.

### Example 61

### 4-{4-[2-(2,6-Dichloro-4-fluorophenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and 2,6-dichloro-4-fluorophenol. LC-MS: Rₜ=0.96 min, ES+=659.17.

### Example 62

### 4-{4-[2-(2,4,6-Trifluorophenoxy)ethoxy]phenyl}-1,2,5,6-tetrahydropyridine-3-carboxylic acid (2-chloro-3-trifluoromethylbenzyl)cyclopropylamide formate salt

According to the general procedures F and B, starting from compound T1 and 2,4,6-trifluorophenol. LC-MS: Rₜ=0.94 min, ES+=625.19.

### Example 63

### 4-{4-[2-(2,6-Difluoro-3-methylphenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,6-difluoro-3-methylphenol. LC-MS: Rₜ=0.94 min, ES+=587.14.

### Example 64

### 4-{4-[2-(4-Chloro-2-methoxyphenoxy)ethoxy]phenyl}-1,2,5,6-tetrahydropyridine-3-carboxylic acid cyclopropyl-(2,3-dichlorobenzyl)amide

According to the general procedures F and B, starting from compound T3 and 4-chloro-2-methoxyphenol. LC-MS: Rₜ=0.93 min, ES+=601.18.

### Example 65

### 4-{4-[2-(2,6-Dichloro-4-methylphenoxy)ethoxy]phenyl}-1,2,5,6-tetrahydropyridine-3-carboxylic acid (6-chlorobenzo[1,3]dioxol-5-ylmethyl)-cyclopropylamide formate salt

According to the general procedures F and B, starting from compound T11 and 2,6-dichloro-4-methylphenol. LC-MS: Rₜ=0.95 min, ES+=629.05.

### Example 66

### 4-{4-[2-(2,6-Dichloro-4-methylphenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2,6-dichloro-4-methylphenol. LC-MS: Rₜ=0.95 min, ES+=630.94

### Example 67

### 4-{4-[2-(2,6-Dichloro-4-methylphenoxy)ethoxy]phenyl}-1,2,5,6-tetrahydropyridine-3-carboxylic acid cyclopropyl-(3-methylbenzyl)amide formate salt

According to the general procedures F and B, starting from compound T7 and 2,6-dichloro-4-methylphenol. LC-MS: Rₜ=0.95 min, ES+=656.12.

### Example 68

### 4-{4-[2-(2,6-Dichloro-4-methylphenoxy)ethoxy]phenyl}-1,2,5,6-tetrahydropyridine-3-carboxylic acid cyclopropyl-(3,5-dimethoxybenzyl)amide formate salt

According to the general procedures F and B, starting from compound T12 and 2,6-dichloro-4-methylphenol. LC-MS: Rₜ=0.93 min, ES+=611.04.

### Example 69

### 4-{4-[2-(2,6-Dichloro-4-methylphenoxy)ethoxy]phenyl}-1,2,5,6-tetrahydropyridine-3-carboxylic acid (3-chlorobenzyl)cyclopropylamide formate salt

According to the general procedures F and B, starting from compound T8 and 2,6-dichloro-4-methylphenol. LC-MS: Rₜ=0.95 min, ES+=587.03.

### Example 70

### 4-{4-[2-(2,6-Dichloro-4-fluorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,6-dichloro-4-fluorophenol. LC-MS: Rₜ=0.94 min, ES+=583.26.

### Example 71

### 4-{4-[2-(2-Chloro-4,5-dimethylphenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and 2-chloro-4,5-dimethylphenol. LC-MS: Rₜ=0.97 min, ES+=633.11.

### Example 72

### 4-{4-[2-(2,6-Dichloro-4-methylphenoxy)ethoxy]phenyl}-1,2,5,6-tetrahydropyridine-3-carboxylic acid (2-chlorobenzyl)ethylamide formate salt

According to the general procedures F and B, starting from compound T9 and 2,6-dichloro-4-methylphenol. LC-MS: Rₜ=0.94 min, ES+=573.07.

### Example 73

### 4-{4-[2-(2,6-Dichloro-4-methylphenoxy)ethoxy]phenyl}-1,2,5,6-tetrahydropyridine-3-carboxylic acid cyclopropyl-(3-methoxybenzyl)amide formate salt

According to the general procedures F and B, starting from compound T13 and 2,6-dichloro-4-methylphenol. LC-MS: Rₜ=0.93 min, ES+=581.09.

### Example 74

### 4-{4-[2-(3-Chloro-2,6-difluorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 3-chloro-2,6-difluorophenol. LC-MS: Rₜ=0.93 min, ES+=567.24.

### Example 75

### 4-{4-[2-(Benzo[1,3]dioxol-5-yloxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and benzo[1,3]dioxol-5-ol. LC-MS: Rₜ=0.94 min, ES+=625.19.

### Example 76

### 4-{4-[2-(2,6-Dichloro-4-methylphenoxy)ethoxy]phenyl}-1,2,5,6-tetrahydropyridine-3-carboxylic acid cyclopropyl-(3-trifluoromethoxybenzyl)amide formate salt

According to the general procedures F and B, starting from compound T6 and 2,6-dichloro-4-methylphenol. LC-MS: Rₜ=0.97 min, ESP+=653.12.

### Example 77

### 4-{4-[2-(2,6-Dichloro-4-fluorophenoxy)ethoxy]phenyl}-1,2,5,6-tetrahydropyridine-3-carboxylic acid cyclopropyl-(3,5-difluorobenzyl)amide formate salt

According to the general procedures F and B, starting from compound T2 and 2,6-dichloro-4-fluorophenol. LC-MS: Rₜ=0.93 min, ES+=593.24.

### Example 78

### 4-{4-[2-(2,6-Dichloro-4-methylphenoxy)ethoxy]phenyl}-1,2,5,6-tetrahydropyridine-3-carboxylic acid cyclopropyl-(3,4-dimethoxybenzyl)amide formate salt

According to the general procedures F and B, starting from compound T14 and 2,6-dichloro-4-methylphenol. LC-MS: Rₜ=0.90 min, ES+=611.06.

### Example 79

### 4-{4-(2-(2,4,6-Trifluorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,4,6-trifluorophenol. LC-MS: Rₜ=0.91 min, ES+=551.30.

### Example 80

### 4-{4-[2-(2-Bromo-5-fluorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2-bromo-5-fluorophenol. LC-MS: Rₜ=0.91 min, ES+=551.30.

### Example 81

### 4-{4-[2-(2,3,6-Trifluorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,3,6-trifluorophenol. LC-MS: Rₜ=0.91 min, ES+=551.12.

### Example 82

### 4-{4-[2-(3-Chloro-2,6-difluorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 3-chloro-2,6-trifluorophenol. LC-MS: Rₜ=0.94 min, ES+=607.14.

### Example 83

### 4-{4-[2-(2,4,6-Trifluorophenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2,4,6-trifluorophenol. LC-MS: Rₜ=0.91 min, ES+=601.15.

### Example 84

### 4-{4-[2-(2-Bromo-5-fluorophenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and 2-bromo-5-fluorophenol. LC-MS: Rₜ=0.95 min, ES+-669.20.

### Example 85

### 4-{4-[2-(Benzo[1,3]dioxol-5-yloxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and benzo[1,3]dioxol-5-ol. LC-MS: Rₜ=0.90 min, ES+=581.17.

### Example 86

### 4-{4-[2-(4-Chloro-2-methoxyphenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and 4-chloro-2-methoxyphenol. LC-MS: Rₜ=0.94 min, ES+=635.16.

### Example 87

### 4-{4-[2-(4-Chloro-2-methoxyphenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 4-chloro-2-methoxyphenol 1. LC-MS: Rₜ=0.92 min, ES+=561.29.

### Example 88

### 4-{4-[2-(2,6-Dichloro-4-methylphenoxy)ethoxy]phenyl}-1,2,5,6-tetrahydropyridine-3-carboxylic acid cyclopropyl-(2-fluoro-5methoxybenzyl)amide formate salt

According to the general procedures F and B, starting from compound T10 and 2,6-dichloro-4-methylphenol. LC-MS: Rₜ=0.94 min, ES+=599.03.

### Example 89

### 4-{4-[2-(2,5-Dichlorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,5-dichlorophenol. LC-MS: Rₜ=0.95 min, ES+=607.20.

### Example 90

### 4-{4-[2-(2-Chloro-4,5-dimethylphenoxy)ethoxy]phenyl}-1,2,5,6-tetrahydropyridine-3-carboxylic acid cyclopropyl-(2,3-dimethylbenzyl)amide

According to the general procedures F and B, starting from compound T5 and 2-chloro-4,5-dimethylphenol. LC-MS: Rₜ=0.95 min, ES+=559.18.

### Example 91

### 4-{4-[2-(2-Chloro-4-trifluoromethylphenoxy)ethoxy]phenyl}-1,2,5,6-tetrahydropyridine-3-carboxylic acid (2-chloro-3-trifluoromethylbenzyl)-cyclopropylamide formate salt

According to the general procedures F and B, starting from compound T1 and 2-chloro-4-trifluoromethylphenol. LC-MS: Rₜ=0.98 min, ES+=673.24.

### Example 92

### 4-{4-[2-(2,6-Difluoro-3-methylphenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and 2,6-difluoro-3-methylphenol. LC-MS: Rₜ=0.95 min, ES+=621.31.

### Example 93

### 4-{4-[2-(2-Chloro-4-trifluoromethylphenoxy)ethoxy]phenyl}-1,2,5,6-tetrahydropyridine-3-carboxylic acid cyclopropyl-(2,3-dimethylbenzyl)-amide formate salt

According to the general procedures F and B, starting from compound T5 and 2-chloro-4-trifluoromethylphenol. LC-MS: Rₜ=0.96 min, ES+=599.30.

### Example 94

### 4-{4-[2-(2,5-Dichlorophenoxy)ethoxy]phenyl}-1,2,5,6-tetrahydropyridine-3-carboxylic acid (2-chloro-3-trifluoromethylbenzyl)cyclopropylamide formate salt

According to the general procedures F and B, starting from compound T1 and 2,5-dichlorophenol. LC-MS: Rₜ=0.96 min, ES+=641.12.

### Example 95

### 4-{4-[2-(2,5-Dichlorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,5-dichlorophenol. LC-MS: Rₜ=0.94 min, ES+=565.23.

### Example 96

### 4-{4-[2-(2-Chloro-4-trifluoromethylphenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2-chloro-4-trifluoromethylphenol phenol. LC-MS: Rₜ=0.97 min, ES+=639.14.

### Example 97

### 4-{4-[2-(2-Bromo-5-fluorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2-bromo-5-fluorophenol. LC-MS: Rₜ=0.94 min, ES+=634.92.

### Example 98

### 4-{4-[2-(2,3-Dichlorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2,3-dichlorophenol. LC-MS: Rₜ=0.94 min, ES+=607.19.

### Example 99

### 4-{4-[2-(2-Chloro-5-fluorophenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 2-chloro-5-fluorophenol. LC-MS: Rₜ=0.93 min, ES+=591.21.

### Example 100

### 4-{4-[2-(2,5-Dichlorophenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2,5-dichlorophenol. LC-MS: Rₜ=0.94 min, ES+=617.11.

### Example 101

### 4-{4-[2-(4-Chloro-2-methylphenoxy)ethoxy]phenyl}-1,2,5,6-tetrahydropyridine-3-carboxylic acid cyclopropyl-(2,3-dichlorobenzyl)amide formate salt

According to the general procedures F and B, starting from compound T3 and 4-chloro-2-methylphenol. LC-MS: Rₜ=0.95 min, ES+=587.22.

### Example 102

### 4-{4-[2-(2,6-Dichloro-4-fluorophenoxy)ethoxy]phenyl}-1,2,5,6-tetrahydropyridine-3-carboxylic acid cyclopropyl-(3-trifluoromethoxybenzyl)amide formate salt

According to the general procedures F and B, starting from compound T6 and 2,6-dichloro-4-fluorophenol. LC-MS: Rₜ=0.95 min, ES+=639.22.

### Example 103

### 4-{4-[2-(2,4,6-Trifluorophenoxy)ethoxy]phenyl}-1,2,5,6-tetrahydropyridine-3-carboxylic acid cyclopropyl-(2-fluoro-5-methoxybenzyl)amide formate salt

According to the general procedures F and B, starting from compound T10 and 2,4,6-trifluorophenol. LC-MS: Rₜ=0.89 min, ES+=571.24.

### Example 104

### 4-{4-[2-(2,6-Dichloro-4-fluorophenoxy)ethoxy]phenyl}-1,2,5,6-tetrahydropyridine-3-carboxylic acid cyclopropyl-(3,5-dimethoxybenzyl)amide formate salt

According to the general procedures F and B, starting from compound T12 and 2,6-dichloro-4-fluorophenol. LC-MS: Rₜ=0.92 min, ES+=615.27.

### Example 105

### 4-{4-[2-(2-Chloro-4,5-dimethylphenoxy)ethoxy]phenyl}-1,2,5,6-tetrahydropyridine-3-carboxylic acid cyclopropyl-(2-fluoro-5-methoxybenzyl)amide formate salt

According to the general procedures F and B, starting from compound T10 and 2-chloro-4,5-dimethylphenol. LC-MS: Rₜ=0.93 min, ES+=579.15.

### Example 106

### 4-{4-[2-(5-Chloro-2-methoxyphenoxy)ethoxy]phenyl}-1,2,5,6-tetrahydropyridine-3-carboxylic acid (2-bromobenzyl)cyclopropylamide formate salt

According to the general procedures F and B, starting from compound T4 and 4-chloro-2-methoxyphenol. LC-MS: Rₜ=0.91 min, ES+=613.21.

### Example 107

### 4-{4-[2-(2,3,6-Trifluorophenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2,3,6-trifluorophenol. LC-MS: Rₜ=0.91 min, ES+=601.09.

### Example 108

### 4-{4-[2-(2-Chloro-4,5-dimethylphenoxy)ethoxy]phenyl}-1,2,5,6-tetrahydropyridine-3-carboxylic acid cyclopropyl-(3,5-dimethoxybenzyl)amide formate salt

According to the general procedures F and B, starting from compound T12 and 2-chloro-4,5-dimethylphenol. LC-MS: Rₜ=0.93 min, ES+=591.18.

### Example 109

### 4-{4-[2-(2-Chloro-4,5-dimethylphenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2-chloro-4,5-dimethylphenol. LC-MS: Rₜ=0.95 min, ES+=611.09.

### Example 110

### 4-{4-[2-(2,6-Dichloro-4-fluorophenoxy)ethoxy]phenyl}-1,2,5,6-tetrahydropyridine-3-carboxylic acid (3-chlorobenzyl)cyclopropylamide formate salt

According to the general procedures F and B, starting from compound T8 and 2,6-dichloro-4-fluorophenol. LC-MS: Rₜ=0.93 min, ES+=589.27.

### Example 111

### 4-{4-[2-(2,6-Difluoro-3-methylphenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,6-difluoro-3-methylphenol. LC-MS: Rₜ=0.92 min, ES+=547.37.

### Example 112

### 4-{4-[2-(2,6-Dichloro-4-fluorophenoxy)ethoxy]phenyl}-1,2,5,6-tetrahydropyridine-3-carboxylic acid cyclopropyl-(2-fluoro-5-methoxybenzyl)amide formate salt

According to the general procedures F and B, starting from compound T10 and 2,6-dichloro-4-fluorophenol. LC-MS: Rₜ=0.92 min, ES+=603.24.

### Example 113

### 4-{4-[2-(2,6-Difluoro-3-methylphenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2,6-difluoro-3-methylphenol. LC-MS: Rₜ=0.92 min, ES+=599.21.

### Example 114

### 4-{4-[2-(2-Fluorophenoxy)ethoxy]phenyl}-1,2,5,6-tetrahydropyridine-3-carboxylic acid (2-chloro-3-trifluoromethylbenzyl)cyclopropylamide formate salt

According to the general procedures F and B, starting from compound T1 and 4-chloro-2-methylphenol. LC-MS: Rₜ=0.96 min, ES+=619.23.

### Example 115

### 4-{4-[2-(2,4,6-Trifluorophenoxy)ethoxy]phenyl}-1,2,5,6-tetrahydropyridine-3-carboxylic acid (6-chlorobenzo[1,3]dioxol-5-ylmethyl)cyclopropylamide formate salt

According to the general procedures F and B, starting from compound T11 and 2,4,6-trifluorophenol. LC-MS: Rₜ=0.91 min, ES+=601.19.

### Example 116

### 4-{4-[2-(3-Chloro-2,6-difluorophenoxy)ethoxy]phenyl}-1,2,5,6-tetrahydropyridine-3-carboxylic acid (6-chlorobenzo[1,3]dioxol-5-ylmethyl)-cyclopropylamide formate salt

According to the general procedures F and B, starting from compound T11 and 2,6-difluoro-3-chlorophenol. LC-MS: Rₜ=0.92 min, ES+=617.19.

### Example 117

### 4-{4-[2-(2,6-Dichloro-4-methylphenoxy)ethoxy]phenyl}-1,2,5,6-tetrahydropyridine-3-carboxylic acid cyclopropyl-(3,5-difluorobenzyl)amide formate salt

According to the general procedures F and B, starting from compound T2 and 2,6-dichloro-4-methylphenol. LC-MS: Rₜ=0.94 min, ES+=587.14.

### Example 118

### 4-{4-[2-(2,4,5-Trichlorophenoxy)ethoxy]phenyl}-1,2,5,6-tetrahydropyridine-3-carboxylic acid (2-chloro-3-trifluoromethylbenzyl)cyclopropylamide formate salt

According to the general procedures F and B, starting from compound T1 and 2,4,5-trichlorophenol. LC-MS: Rₜ=0.98 min, ES+=675.22.

### Example 119

### 4-{4-[2-(2-Chloro-5-fluorophenoxy)ethoxy]phenyl}-1,2,5,6-tetrahydropyridine-3-carboxylic acid (2-chloro-3-trifluoromethylbenzyl)cyclopropyl-amide formate salt

According to the general procedures F and B, starting from compound T1 and 2-chloro-5-fluorophenol. LC-MS: Rₜ=0.94 min, ES+=623.29.

### Example 120

### 4-{4-[2-(2,3-Dichlorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,3-dichlorophenol. LC-MS: Rₜ=0.93 min, ES+=565.28.

### Example 121

### 4-{4-[2-(2,6-Dichloro-4-fluorophenoxy)ethoxy]phenyl}-1,2,5,6-tetrahydropyridine-3-carboxylic acid (2-chlorobenzyl)ethylamide formate salt

According to the general procedures F and B, starting from compound T9 and 2,6-dichloro-4-fluorophenol. LC-MS: Rₜ=0.93 min, ES+=579.15.

### Example 122

### 4-{4-[2-(2,4,5-Trichlorophenoxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and 2,4,5-trichlorophenol. LC-MS: Rₜ=0.96 min, ES+=599.32.

### Example 123

### 4-{4-[2-(3-Chloro-2,6-difluorophenoxy)ethoxy]phenyl}-1,2,5,6-tetrahydropyridine-3-carboxylic acid (2-bromobenzyl)cyclopropylamide formate salt

According to the general procedures F and B, starting from compound T4 and 3-chloro-2,3-difluorophenol. LC-MS: Rₜ=0.93 min, ES+=619.11.

### Example 124

### 4-{4-[2-(Benzo[1,3]dioxol-5-yloxy)ethoxy]phenyl}-1,2,5,6-tetrahydropyridine-3-carboxylic acid cyclopropyl-(2,3-dimethylbenzyl)amide formate salt

According to the general procedures F and B, starting from compound T5 and benzo[1,3]dioxol-5-ol. LC-MS: Rₜ=0.89 min, ES+=541.32.

### Example 125

### 4-{4-[2-(2-Chloro-4-trifluoromethylphenoxy)ethoxy]phenyl}-1,2,5,6-tetrahydropyridine-3-carboxylic acid cyclopropyl-(3,5-dimethoxybenzyl)-amide formate salt

According to the general procedures F and B, starting from compound T12 and 2-chloro-4-trifluoromethylphenol. LC-MS: Rₜ=0.94 min, ES+=631.27.

### Example 126

### 4-{4-[2-(2,4,6-Trifluorophenoxy)ethoxy]phenyl}-1,2,5,6-tetrahydropyridine-3-carboxylic acid cyclopropyl-(3,5-dimethoxybenzyl)amide formate salt

According to the general procedures F and B, starting from compound T12 and 2,4,6-trifluorophenol. LC-MS: Rₜ=0.89 min, ES+=583.24.

### Example 127

### 4-{4-[2-(2,4,6-Trifluorophenoxy)ethoxy]phenyl}-1,2,5,6-tetrahydropyridine-3-carboxylic acid (2-chlorobenzyl)ethylamide formate salt

According to the general procedures F and B, starting from compound T9 and 2,4,6-trifluorophenol. LC-MS: Rₜ=0.90 min, ES+=545.24.

### Example 128

### 4-{4-[2-(2-Chloro-4-trifluoromethylphenoxy)ethoxy]phenyl}-1,2,5,6-tetrahydropyridine-3-carboxylic acid cyclopropyl-(2-fluoro-5-methoxy-benzyl)amide formate salt

According to the general procedures F and B, starting from compound T10 and 2-chloro-4-trifluoromethylphenol. LC-MS: Rₜ=0.94 min, ES+=619.26.

### Example 129

### 4-{4-[2-(2,6-Dichloro-4-fluorophenoxy)ethoxy]phenyl}-1,2,5,6-tetrahydro-pyridine-3-carboxylic acid (6-chlorobenzo[1,3]dioxol-5-ylmethyl)-cyclopropylamide formate salt

According to the general procedures F and B, starting from compound T11 and 2,6-dichloro-4-fluorophenol. LC-MS: Rₜ=0.94 min, ES+=633.25.

### Example 130

### 4-{4-[2-(4-Chloro-2-methoxyphenoxy)ethoxy]phenyl}-1,2,5,6-tetrahydro-pyridine-3-carboxylic acid cyclopropyl-(3-methoxybenzyl)amide formate salt

According to the general procedures F and B, starting from compound T13 and 4-chloro-2-methoxyphenol. LC-MS: Rₜ=0.89 min, ES+=563.26

### Example 131

### 4-{4-[2-(2-Chloro-4-trifluoromethylphenoxy)ethoxy]phenyl}-1,2,5,6-tetrahydropyridine-3-carboxylic acid (2-bromobenzyl)cyclopropylamide formate salt

According to the general procedures F and B, starting from compound T4 and 2-chloro-4-trifluoromethylphenol. LC-MS: Rₜ=0.96 min, ES+=651.16.

### Example 132

### 4-{4-[2-(2-Chloro-4-trifluoromethylphenoxy)ethoxy]phenyl}-1,2,5,6-tetrahydropyridine-3-carboxylic acid cyclopropyl-(3-methoxybenzyl)amide formate salt

According to the general procedures F and B, starting from compound T13 and 2-chloro-4-trifluoromethylphenol. LC-MS: Rₜ=0.93 min, ES+=601.26.

### Example 133

### 4-{4-[2-(2,3,6-Trifluorophenoxy)ethoxy]phenyl}-1,2,5,6-tetrahydropyridine-3-carboxylic acid (2-chlorobenzyl)ethylamide formate salt

According to the general procedures F and B, starting from compound T9 and 2,3,6-trifluorophenol. LC-MS: Rₜ=0.90 min, ES+=545.04.

### Example 134

### 4-{4-[2-(3-Chloro-2,6-difluorophenoxy)ethoxy]phenyl}-1,2,5,6-tetrahydro-pyridine-3-carboxylic acid cyclopropyl-(2-fluoro-5-methoxybenzyl)amide formate salt

According to the general procedures F and B, starting from compound T10 and 2,6-difluoro-3-chlorophenol. LC-MS: Rₜ=0.91 min, ES+=587.21.

### Example 135

### 4-{4-[2-(2-Bromo-5-fluorophenoxy)ethoxy]phenyl}-1,2,5,6-tetrahydro-pyridine-3-carboxylic acid cyclopropyl-(2-fluoro-5-methoxybenzyl)amide formate salt

According to the general procedures F and B, starting from compound T12 (50 mg) and 2-bromo-5-fluorophenol. LC-MS: Rₜ=0.90 min, ES+=613.03.

### Example 136

### 4-{4-[2-(2,5-Dichlorophenoxy)ethoxy]phenyl)-1,2,5,6-tetrahydropyridine-3-carboxylic acid cyclopropyl-(3,5-dimethoxybenzyl)amide formate salt

According to the general procedures F and B, starting from compound T12 and 2,5-dichlorophenol. LC-MS: Rₜ=0.92 min, ES+=597.23.

### Example 137

### 4-{4-[2-(2-Chloro-4,5-dimethylphenoxy)ethoxy]phenyl}-1,2,5,6-tetrahydro-pyridine-3-carboxylic acid cyclopropyl-(3-methoxybenzyl)amide formate salt

According to the general procedures F and B, starting from compound T13 and 2-chloro-4,5-dimethylphenol. LC-MS: Rₜ=0.92 min, ES+=561.14.

### Example 138

### 4-{4-[2-(4-Chloro-2-methylphenoxy)ethoxy]phenyl}-1,2,5,6-tetrahydro-pyridine-3-carboxylic acid cyclopropyl-(2,3-dimethylbenzyl)amide formate salt

According to the general procedures F and B, starting from compound T4 and 4-chloro-2-methylphenol. LC-MS: Rₜ=0.94 min, ES+=597.20.

### Example 139

### 4-{4-[2-(4-Chloro-2-methoxyphenoxy)ethoxy]phenyl}-1,2,5,6-tetrahydro-pyridine-3-carboxylic acid (6-chlorobenzo[1,3]dioxol-5-ylmethyl)-cyclopropylamide formate salt

According to the general procedures F and B, starting from compound T11 and 4-chloro-2-methoxyphenol. LC-MS: Rₜ=0.91 min, ES+=611.23.

### Example 140

### 4-{4-[2-(2-Bromo-5-fluorophenoxy)ethoxy]phenyl}-1,2,5,6-tetrahydro-pyridine-3-carboxylic acid (2-bromobenzyl)cyclopropylamide formate salt

According to the general procedures F and B, starting from compound T4 and 2-bromo-4-fluorophenol. LC-MS: Rₜ=0.92 min. ES+=645.08.

### Example 141

### 4-{4-[2-(3-Chloro-2,6-difluorophenoxy)ethoxy]phenyl}-1,2,5,6-tetrahydro-pyridine-3-carboxylic acid cyclopropyl-(3-methoxybenzyl)amide

According to the general procedures F and B, starting from compound T13 and 2,6-difluoro-3-chlorophenol. LC-MS: Rₜ=0.90 min, ES+=569.23.

### Example 142

### 4-{4-[2-(2-Chloro-4-trifluoromethylphenoxy)ethoxy]phenyl}-1,2,5,6-tetrahydro-pyridine-3-carboxylic acid (6-chlorobenzo[1,3]dioxol-5-ylmethyl)-cyclopropylamide formate salt

According to the general procedures F and B, starting from compound T11 and 2-chloro-4-trifluoromethylphenol. LC-MS: Rₜ=0.95 min, ES+=649.22.

### Example 143

### 4-{4-[2-(2-Chloro-4,5-dimethylphenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 2-chloro-4,5-dimethylphenol. LC-MS: Rₜ=0.94 min, ES+=565.28.

### Example 144

### 4-{4-[2-(2,6-Dichloro-4-methylphenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 2,6-dichloro-4-methylphenol. LC-MS: Rₜ=0.95 min, ES+=587.22.

### Example 145

### 4-{4-[2-(2,4,5-Trichlorophenoxy)ethoxy]phenyl}-1,2,5,6-tetrahydropyridine-3-carboxylic acid (2-chlorobenzyl)cyclopropylamide formate salt

According to the general procedures F and B, starting from compound T15 and 2,4,5-trichlorophenol. LC-MS: Rₜ=0.95 min, ES+=607.19.

### Example 146

### 4-{4-[2-(2-Chloro-5-fluorophenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 2-chloro-5-fluorophenol. LC-MS: Rₜ=0.91 min, ES+=555.26.

### Example 147

### 4-{4-[2-(2-Chloro-3,6-difluorophenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 2-chloro-3,6-difluorophenol. LC-MS: Rₜ=0.91 min, ES+=573.21.

### Example 148

### 4-{4-[2-(2-Chloro-6-methylphenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 2-chloro-6-methylphenol. LC-MS: Rₜ=0.92 min, ES+=551.30.

### Example 149

### 4-{4-[2-(2,3-Dichlorophenoxy)ethoxy]phenyl)-1,2,5,6-tetrahydro-pyridine-3-carboxylic acid (2-chlorobenzyl)cyclopropylamide formate salt

According to the general procedures F and B, starting from compound T15 and 2,3-dichlorophenol. LC-MS: Rₜ=0.92 min, ES+=571.21.

### Example 150

### 4-{4-[2-(2,6-Dichloro-4-fluorophenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 2,6-dichloro-4-fluorophenol. LC-MS: Rₜ=0.93 min, ES+=589.20.

### Example 151

### 4-{4-[2-(3-Chloro-2,6-difluorophenoxy)ethoxy]phenyl}-1,2,5,6-tetrahydro-pyridine-3-carboxylic acid (2-chlorobenzyl)cyclopropylamide formate salt

According to the general procedures F and B, starting from compound T15 and 3-chloro-2,6-difluorophenol. LC-MS: Rₜ=0.91 min, ES+=573.24.

### Example 152

### 4-{4-[2-(2,4,6-Trifluorophenoxy)ethoxy]phenyl}-1,2,5,6-tetrahydropyridine-3-carboxylic acid (2-chlorobenzyl)cyclopropylamide formate salt

According to the general procedures F and B, starting from compound T15 and 2,4,6-trifluorophenol. LC-MS: Rₜ=0.90 min, ES+=557.28.

### Example 153

### 4-{4-[2-(2,5-Dichlorophenoxy)ethoxy]phenyl}-1,2,5,6-tetrahydropyridine-3-carboxylic acid (2-chlorobenzyl)cyclopropylamide formate salt

According to the general procedures F and B, starting from compound T15 and 2,5-dichlorophenol. LC-MS: Rₜ=0.93 min, ES+=573.21.

### Example 154

### 4-{4-[2-(2,6-Dichlorophenoxy)ethoxy]phenyl}-1,2,5,6-tetrahydropyridine-3-carboxylic acid (2-chlorobenzyl)cyclopropylamide formate salt

According to the general procedures F and B, starting from compound T15 and 2,6-dichlorophenol. LC-MS: Rₜ=0.92 min, ES+=573.20.

The following assay was carried out in order to determine the activity of the compounds of general formula I and their salts.

Inhibition of Human Recombinant Renin by the Compounds of the Invention

The enzymatic in vitro assay was performed in 384-well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:

human recombinant renin (0.16 ng/mL)

synthetic human angiotensin(1-14) (0.5 μM)

hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384-well plates (Nunc). 5 μL of the incubates or standards were transferred to immuno plates which were previously coated with a covalent complex of Ang I and bovine serum albumin (Ang I-BSA). 75 μL of Ang I-antibodies in assay buffer above including 0.01% Tween 20 were added and a primary incubation made at 4° C. overnight. The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at rt. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀). The IC₅₀-values of all compounds tested are below 100 nM. However selected compounds exhibit a very good bioavailibility and are metabolically more stable than prior art compounds.

1. A compound of the general formula I 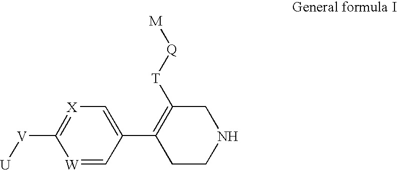
wherein
X and W represent independently a nitrogen atom or a —CH— group;
V represents —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂-CH₂—; —CH₂-A-CH₂—CH₂—B—CH₂—; —CH₂-A-CH₂—CH₂—CH₂—B—; —CH₂—CH₂-A-CH₂—CH₂—B—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; —COO—; —(CH₂)_{P}OCONR¹; —(CH₂)_{P}N(R^{1′})CONR¹—;
Q represents lower alkylene; lower alkenylene;
M represents hydrogen; cycloalkyl; aryl; heterocyclyl; heteroaryl;
R¹ and R^{1′} independently represent hydrogen; lower alkyl; lower alkenyl; lower alkinyl; cycloalkyl; aryl; cycloalkyl-lower alkyl;
p is the integer1, 2, 3 or 4;
r is the integer 3, 4, 5, or 6;
s is the integer 2, 3, 4, or 5;
t is the integer 1, 2, 3, or 4;
u is the integer 1, 2, or 3; and
v is the integer 2, 3, or 4;
and optically pure enantiomers, mixtures of enantiomers, diastereomers, mixtures of diastereomers, diastereomeric racemates, mixtures of diastereomeric racemates, and the meso-form; and pharmaceutically acceptable salts, solvent complexes and morphological forms, of said compound.

2. The compound of claim 1, wherein
T represents —CONR¹—;
Q represents methylene; and
M represents hydrogen; aryl or heteroaryl;
and optically pure enantiomers, mixtures of enantiomers, diastereomers, mixtures of diastereomers, diastereomeric racemates, mixtures of diastereomeric racemates, and the meso-form; and pharmaceutically acceptable salts, solvent complexes and morphological forms, of said compound.

3. The compound of claim 1, wherein
V represents —CH₂CH₂O—;—CH₂CH₂CH₂O—; —OCH₂CH₂O—;
and optically pure enantiomers, mixtures of enantiomers, diastereomers, mixtures of diastereomers, diastereomeric racemates, mixtures of diastereomeric racemates, and the meso-form; and pharmaceutically acceptable salts, solvent complexes and morphological forms, of said compound.

4. The compound of claim 1, wherein
X and W represent —CH—;
and optically pure enantiomers, mixtures of enantiomers, diastereomers, mixtures of diastereomers, diastereomeric racemates, mixtures of diastereomeric racemates, and the meso-form; pharmaceutically acceptable salts, solvent complexes and morphological forms, and said compound.

5. The compound of claim 1, wherein
U represents a mono-, di-, or trisubstituted phenyl and the substituents are independently halogen, lower alkyl, lower alkoxy, trifluoromethyl, trifluoromethoxy;
and optically pure enantiomers, mixtures of enantiomers, diastereomers, mixtures of diastereomers, diastereomeric racemates, mixtures of diastereomeric racemates, and the meso-form; and pharmaceutically acceptable salts, solvent complexes and morphological forms, of said compound.

6. A compound selected from the group consisting of
4-{4-[3-(2-methoxybenzyloxy)propoxy]phenyl}-1,2,5,6-tetrahydropyridine-3-carboxylic acid [2-(2-chlorophenyl)ethyl]methylamide;
4-{4-[3-(2-bromo-5-fluorophenoxy)propyl]phenyl}-1,2,5,6-tetrahydropyridi-ne-3-carboxylic acid [2-(2-chlorophenyl)ethyl]methylamide;
4-{4-[3-(2-bromo-5-fluorophenoxy)propyl]phenyl}-1,2,5,6-tetrahydropyridi-ne-3-carboxylic acid 2-phenethylmethylamide;
4-{4-[3-(2-bromo-5-fluorophenoxy)propyl]phenyl}-1,2,5,6-tetrahydropyridi-ne-3-carboxylic acid (2-chlorobenzyl)methylamide;
4-{4-[3-(2-bromo-5-fluorophenoxy)propyl]phenyl}-1,2,5,6-tetrahydropyridi-ne-3-carboxylic acid (2-chlorobenzyl)cyclopropylamide;
4-{4-[3-(2-chlorophenoxy)propyl]phenyl}-1,2,5,6-tetrahydropyridine-3-carboxylic acid [2-(2-chlorophenyl)ethyl]methylamide;
4-{4-[3-(2-chlorophenoxy)propyl]phenyl}-1,2,5,6-tetrahydropyridine-3-carboxylic acid 2-phenethylmethylamide;
4-{4-[3-(2-chlorophenoxy)propyl]phenyl}-1,2,5,6-tetrahydropyridine-3-carboxylic acid (2-chlorobenzyl)methylamide;
4-{4-[3-(2-chlorophenoxy)propyl]phenyl}-1,2,5,6-tetrahydropyridine-3-carboxylic acid (2-chlorobenzyl)cyclopropylamide;
4-{4-[3-(2,5-difluorophenoxy)propyl]phenyl}-1,2,5,6-tetrahydropyridine-3-carboxylic acid [2-(2-chlorophenyl)ethyl]methylamide;
4-{4-[3-(2,5-difluorophenoxy)propyl]phenyl}-1,2,5,6-tetrahydropyridine-3-carboxylic acid 2-phenethylmethylamide;
4-{4-[3-(2,5-difluorophenoxy)propyl]phenyl}-1,2,5,6-tetrahydropyridine-3-carboxylic acid (2-chlorobenzyl)methylamide;
4-{4-[3-(2,5-difluorophenoxy)propyl]phenyl}-1,2,5,6-tetrahydropyridine-3-carboxylic acid (2-chlorobenzyl)cyclopropylamide;
4-{4-[3-(2-bromo-5-fluorophenoxy)propyl]phenyl}-1,2,5,6-tetrahydropyridine-3-carboxylic acid (2-chlorobenzyl)cyclopropylamide;
4-{4-[3-(2,3,6-trifluorophenoxy)propyl]phenyl}-1,2,5,6-tetrahydropyridine-3-carboxylic acid (2-chlorobenzyl)cyclopropylamide;
4-{4-[3-(2,3,6-trifluorophenoxy)propyl]phenyl}-1,2,5,6-tetrahydropyridine-3-carboxylic acid (2-chlorobenzyl)ethylamide;
4-{4-[3-(2,3,6-trifluorophenoxy)propyl]phenyl}-1,2,5,6-tetrahydropyridine-3-carboxylic acid cyclopropyl-(2-fluorobenzyl)amide;
4-{4-[3-(2,3,6-trifluorophenoxy)propyl]phenyl}-1,2,5,6-tetrahydropyridine-3-carboxylic acid cyclopropyl-(3-trifluoromethylbenzyll)amide;
4-{4-[3-(2,3,6-trifluorophenoxy)propyl]phenyl}-1,2,5,6-tetrahydropyridine-3-carboxylic acid cyclopropyl-(2-methylbenzyl)amide;
4-{4-[3-(2,3,6-trifluorophenoxy)propyl]phenyl}-1,2,5,6-tetrahydropyridine-3-carboxylic acid cyclopropyl-[2-(4-methoxyphenoxy)ethyl]amide;
4-{4-[3-(2,3,6-trifluorophenoxy)propyl]phenyl}-1,2,5,6-tetrahydropyridine-3-carboxylic acid cyclopropyl-[2-(3-methoxyphenoxy)ethyl]amide;
4-{4-[3-(2,3,6-trifluorophenoxy)propyl]phenyl}-1,2,5,6-tetrahydropyridine-3-carboxylic acid cyclopropyl-(2-m-tolyloxyethyl)amide;
4-{4-[3-(2,3,6-trifluorophenoxy)propyl]phenyl}-1,2,5,6-tetrahydropyridine-3-carboxylic acid [2-(2-chlorophenyl)ethyl]cyclopropylamide;
4-{4-[3-(2,3,6-trifluorophenoxy)propyl]phenyl}-1,2,5,6-tetrahydropyridine-3-carboxylic acid cyclopropyl-[2-(4-fluorophenyl)ethyl]amide;
4-{4-[3-(2,3,6-trifluorophenoxy)propyl]phenyl}-1,2,5,6-tetrahydropyridine-3-carboxylic acid cyclopropyl-(2-o-tolylethyl)amide;
4-{4-[3-(2,3,6-trifluorophenoxy)propyl]phenyl}-1,2,5,6-tetrahydropyridine-3-carboxylic acid cyclopropyl-(3,5-dimethoxybenzyl)amide;
4-{4-[3-(2,3,6-trifluorophenoxy)propyl]phenyl}-1,2,5,6-tetrahydropyridine-3-carboxylic acid cyclopropyl-(2-p-tolylethyl)amide;
4-{4-[2-(2,3,5-trimethylphenoxy)ethyl]phenyl}-1,2,5,6-tetrahydropyridine-3-carboxylic acid cyclopropyl-(3-trifluoromethylbenzyl)amide;
4-{4-[2-(2,3,5-trimethylphenoxy)ethyl]phenyl}-1,2,5,6-tetrahydropyridine-3-carboxylic acid cyclopropyl-(2-methylbenzyl)amide;
4-{4-[2-(2,3,5-trimethylphenoxy)ethyl]phenyl}-1,2,5,6-tetrahydropyridine-3-carboxylic acid cyclopropylphenethylamide;
4-{4-[3-(2-bromo-5-fluorophenoxy)propyl]phenyl}-1,2,5,6-tetrahydropyridine-3-carboxylic acid (2-chlorobenzyl)ethylamide;
4-{4-[3-(2-bromo-5-fluorophenoxy)propyl]phenyl}-1,2,5,6-tetrahydropyridine-3-carboxylic acid cyclopropyl-(2-methylbenzyl)amide;
4-{4-[3-(2-bromo-5-fluorophenoxy)propyl]phenyl}-1,2,5,6-tetrahydropyridine-3-carboxylic acid cyclopropyl-[2-(4-methoxyphenoxy)ethyl]amide;
4-{4-[3-(2-bromo-5-fluorophenoxy)propyl]phenyl}-1,2,5,6-tetrahydropyridine-3-carboxylic acid cyclopropylphenethylamide;
4-{4-[3-(2-bromo-5-fluorophenoxy)propyl]phenyl}-1,2,5,6-tetrahydropyridine-3-carboxylic acid cyclopropyl-(2-o-tolylehtyl)amide;
4-{4-[3-(2-bromo-5-fluorophenoxy)propyl]phenyl}-1,2,5,6-tetrahydropyridine-3-carboxylic acid cyclopropyl-(3,5-dimethoxybenzyl)amide;
4-{4-[3-(2-bromo-5-fluorophenoxy)propyl]phenyl}-1,2,5,6-tetrahydropyridine-3-carboxylic acid cyclopropyl-(2-p-tolylethyl)amide;
4-{4-[2-(2-chloro-4,5-dimethylphenoxy)ethoxy]phenyl}-1,2,5,6-tetrahydro-pyridine-3-carboxylic acid(2-chlorobenzyl)cyclopropylamide;
4-{4-[3-(2,3,6-trifluorophenoxy)propyl]phenyl}-1,2,5,6-tetrahyrdopyridine-3-carboxylic acid cyclopropyl-(2-fluoro-5-methoxybenzyl)amide;
4-{4-[3-(2,3,6-trifluorophenoxy)propyl]phenyl}-1,2,5,6-tetrahydropyridine-3-carboxylic acid cyclopropyl-(3-methoxybenzyl)amide;
4-{4-[3-(2,3,6-trifluorophenoxy)propyl]phenyl}-1,2,5,6-tetrahydropyridine-3-carboxylic acid cyclopropyl-(3,4-dimethoxybenzyl)amide;
4-{4-[3-(2,3,6-trifluorophenoxy)propyl]phenyl}-1,2,5,6-tetrahydropyridine-3-carboxylic acid (2-chloro-3-trifluoromethylbenzyl)cyclopropylamide;
4-{4-[3-(2,3,6-trifluorophenoxy)propyl]phenyl}-1,2,5,6-tetrahydropyridine-3-carboxylic acid (6-chlorobenzo[1,3]dioxol-5-ylmethyl)cyclopropylamide;
4-{4-[3-(2,3,6-trifluorophenoxy)propyl]phenyl}-1,2,5,6-tetrahydropyridine-3-carboxylic acid (2-chloro-6-fluorobenzyl)cyclopropylamide;
4-{4-[3-(2,3,6-trifluorophenoxy)propyl]phenyl}-1,2,5,6-tetrahydropyridine-3-carboxylic acid (2-bromobenzyl)cyclopropylamide;
4-{4-[3-(2,3,6-trifluorophenoxy)propyl]phenyl}-1,2,5,6-tetrahydropyridine-3-carboxylic acid cyclopropyl-(2,3-dimethylbenzyl)amide;
4-{4-[3-(2,3,6-trifluorophenoxy)propyl]phenyl}-1,2,5,6-tetrahydropyridine-3-carboxylic acid cyclopropyl-(3-fluoro-2-methylbenzyl)amide;
4-{4-[3-(2,3,6-trifluorophenoxy)propyl]phenyl}-1,2,5,6-tetrahydropyridine-3-carboxylic acid cyclopropyl-(2,3-dichlorobenzyl)amide;
4-{4-[3-(2,3,6-trifluorophenoxy)propyl]phenyl}-1,2,5,6-tetrahydropyridine-3-carboxylic acid cyclopropyl-(3-methylbenzyl)amide;
4-{4-[3-(2,3,6-trifluorophenoxy)propyl]phenyl}-1,2,5,6-tetrahydropyridine-3-carboxylic acid cyclopropyl-(2,3-difluorobenzyl)amide;
4-{4-[3-(2,3,6-trifluorophenoxy)propyl]phenyl}-1,2,5,6-tetrahydropyridine-3-carboxylic acid (3-chlorobenzyl)cyclopropylamide;
4-{4-[2-(2,6-dichloro-4-methylphenoxy)ethoxy]phenyl}-1,2,5,6-tetrahydro-pyridine-3-carboxylic acid cyclopropyl-(2,3-dichlorobenzyl)amide;
4-{4-[2-(2,6-dichloro-4-methylphenoxy)ethoxy]phenyl}-1,2,5,6-tetrahydro-pyridine-3-carboxylic acid (2-chloro-3-trifluoromethylbenzyl)cyclopropylamide;
4-{4-[2-(2,6-dichloro-4-methylphenoxy)ethoxy]phenyl}-1,2,5,6-tetrahydro-pyridine-3-carboxylic acid cyclopropyl-(2,3-dimethylbenzyl)amide;
4-{4-[2-(2,3,6-trifluorophenoxy)ethoxy]phenyl}-1,2,5,6-tetrahydropyridine-3-carboxylic acid (2-chloro-3-trifluoromethylbenzyl)cyclopropylamide;
4-{4-[2-(2,6-dichloro-4-fluorophenoxy)ethoxy]phenyl}-1,2,5,6-tetrahydro-pyridine-3-carboxylic acid (2-bromobenzyl)cyclopropylamide;
4-{4-[2-(2,4,6-trifluorophenoxy)ethoxy]phenyl}-1,2,5,6-tetrahydropyridine-3-carboxylic acid cyclopropyl-(2,3-dichlorobenzyl)amide;
4-{4-[2-(2,6-dichloro-4-fluorophenoxy)ethoxy]phenyl}-1,2,5,6-tetrahydro-pyridine-3-carboxylic acid cyclopropyl-(2,3-dichlorobenzyl)amide;
4-{4-[2-(2-chloro-4,5-dimethylphenoxy)ethoxy]phenyl}-1,2,5,6-tetrahydro-pyridine-3-carboxylic acid cyclopropyl-(2,3-dichlorobenzyl)amide;
4-{4-[2-(2,3,6-trifluorophenoxy)ethoxy]phenyl}-1,2,5,6-tetrahydropyridine-3-carboxylic acid cyclopropyl-(2,3-dichlorobenzyl)amide;
4-{4-[2-(2,6-dichloro-4-fluorophenoxy)ethoxy]phenyl}-1,2,5,6-tetrahydro-pyridine-3-carboxylic acid (2-chloro-3-trifluoromethylbenzyl)cyclopropylamide;
4-{4-[2-(2,4,6-trifluorophenoxy)ethoxy]phenyl}-1,2,5,6-tetrahydropyridine-3-carboxylic acid (2-chloro-3-trifluoromethylbenzyl)cyclopropylamide;
4-{4-[2-(2,6-difluoro-3-methylphenoxy)ethoxy]phenyl}-1,2,5,6-tetrahydro-pyridine-3-carboxylic acid cyclopropyl-(2,3-dichlorobenzyl)amide;
4-{4-[2-(4-chloro-2-methoxyphenoxy)ethoxy]phenyl}-1,2,5,6-tetrahydro-pyridine-3-carboxylic acid cyclopropyl-(2,3-dichlorobenzyl)amide;
4-{4-[2-(2,6-dichloro-4-methylphenoxy)ethoxy]phenyl}-1,2,5,6-tetrahydro-pyridine-3-carboxylic acid (6-chlorobenzo[1,3]dioxol-5-ylmethyl)-cyclopropylamide;
4-{4-[2-(2,6-dichloro-4-methylphenoxy)ethoxy]phenyl}-1,2,5,6-tetrahydro-pyridine-3-carboxylic acid (2-bromobenzyl)cyclopropylamide;
4-{4-[2-(2,6-dichloro-4-methylphenoxy)ethoxy]phenyl}-1,2,5,6-tetrahydro-pyridine-3-carboxylic acid cyclopropyl-(3-methylbenzyl)amide;
4-{4-[2-(2,6-dichloro-4-methylphenoxy)ethoxy]phenyl}-1,2,5,6-tetrahydro-pyridine-3-carboxylic acid cyclopropyl-(3,5-dimethoxybenzyl)amide;
4-{4-[2-(2,6-dichloro-4-methylphenoxy)ethoxy]phenyl}-1,2,5,6-tetrahydro-pyridine-3-carboxylic acid (3-chlorobenzyl)cyclopropylamide;
4-{4-[2-(2,6-dichloro-4-fluorophenoxy)ethoxy]phenyl}-1,2,5,6-tetrahydro-pyridine-3-carboxylic acid cyclopropyl-(2,3-dimethylbenzyl)amide;
4-{4-[2-(2-chloro-4,5-dimethylphenoxy)ethoxy]phenyl}-1,2,5,6-tetrahydro-pyridine-3-carboxylic acid (2-chloro-3-trifluoromethylbenzyl)cyclopropylamide;
4-{4-[2-(2,6-dichloro-4-methylphenoxy)ethoxy]phenyl}-1,2,5,6-tetrahydro-pyridine-3-carboxylic acid (2-chlorobenzyl)ethylamide;
4-{4-[2-(2,6-dichloro-4-methylphenoxy)ethoxy]phenyl}-1,2,5,6-tetrahydro-pyridine-3-carboxylic acid cyclopropyl-(3-methoxybenzyl)amide;
4-{4-[2-(3-chloro-2,6-difluorophenoxy)ethoxy]phenyl}-1,2,5,6-tetrahydro-pyridine-3-carboxylic acid cyclopropyl-(2,3-dimethylbenzyl)amide;
4-{4-[2-(benzo[1,3]dioxol-5-yloxy)ethoxy]phenyl}-1,2,5,6-tetrahydro-pyridine-3-carboxylic acid (2-chloro-3-trifluoromethylbenzyl)cyclopropylamide;
4-{4-[2-(2,6-dichloro-4-methylphenoxy)ethoxy]phenyl}-1,2,5,6-tetrahydro-pyridine-3-carboxylic acid cyclopropyl-(3-trifluoromethoxybenzyl)amide;
4-{4-[2-(2,6-dichloro-4-fluorophenoxy)ethoxy]phenyl}-1,2,5,6-tetrahydro-pyridine-3-carboxylic acid cyclopropyl-(3,5-difluorobenzyl)amide;
4-{4-[2-(2,6-dichloro-4-methylphenoxy)ethoxy]phenyl}-1,2,5,6-tetrahydro-pyridine-3-carboxylic acid cyclopropyl-(3,4-dimethoxybenzyl)amide;
4-{4-[2-(2,4,6-trifluorophenoxy)ethoxy]phenyl}-1,2,5,6-tetrahydropyridine-3-carboxylic acid cyclopropyl-(2,3-dimethylbenzyl)amide;
4-{4-[2-(2-bromo-5-fluorophenoxy)ethoxy]phenyl}-1,2,5,6-tetrahydro-pyridine-3-carboxylic acid cyclopropyl-(2,3-dimethylbenzyl)amide;
4-{4-[2-(2,3,6-trifluorophenoxy)ethoxy]phenyl}-1,2,5,6-tetrahydropyridine-3-carboxylic acid cyclopropyl-(2,3-dimethylbenzyl)amide;
4-{4-[2-(3-chloro-2,6-difluorophenoxy)ethoxy]phenyl}-1,2,5,6-tetrahydro-pyridine-3-carboxylic acid cyclopropyl-(2,3-dichlorobenzyl)amide;
4-{4-[2-(2,4,6-trifluorophenoxy)ethoxy]phenyl}-1,2,5,6-tetrahydropyridine-3-carboxylic acid (2-bromobenzyl)cyclopropylamide;
4-{4-[2-(2-bromo-5-fluorophenoxy)ethoxy]phenyl}-1,2,5,6-tetrahydropyridine-3-carboxylic acid (2-chloro-3-trifluoromethylbenzyl)cyclopropylamide;
4-{4-[2-(benzol[1,3]dioxol-5-yloxy)ethoxy]phenyl}-1,2,5,6-tetrahydro-pyridine-3-carboxylic acid cyclopropyl-(2,3-dichlorobenzyl)amide;
4-{4-[2-(4-chloro-2-methoxyphenoxy)ethoxy]phenyl}-1,2,5,6-tetrahydro-pyridine-3-carboxylic acid (2-chloro-3-trifluoromethylbenzyl)cyclopropylamide;
4-{4-[2-(4-chloro-2-methoxyphenoxy)ethoxy]phenyl}-1,2,5,6-tetrahydro-pyridine-3-carboxylic acid cyclopropyl(2,3-dimethylbenzyl)amide;
4-{4-[2-(2,6-dichloro-4-methoxyphenoxy)ethoxy]phenyl}-1,2,5,6-tetrahydro-pyridine-3-carboxylic acid cyclopropyl-(2-fluoro-5-methoxybenzyl)amide;
4-{4-[2-(2,5-dichlorophenoxy)ethoxy]phenyl}-1,2,5,6-tetrahydropyridine-3-carboxylic acid cyclopropyl-(2,3-dichlorobenzyl)amide;
4-{4-[2-(2-chloro-4,5-dimethylphenoxy)ethoxy]phenyl}-1,2,5,6-tetrahydro-pyridine-3-carboxylic acid cyclopropyl-(2,3-dimethoxybenzyl)amide;
4-{4-[2-(2-chloro-4-trifluoromethylphenoxy)ethoxy]phenyl}-1,2,5,6-tetrahydro-pyridine-3-carboxylic acid (2-chloro-3-trifluoromethylbenzyl)cyclopropylamide;
4-{4-[2-(2,6-difluoro-3-methylphenoxy)ethoxy]phenyl}-1,2,5,6-tetrahydro-pyridine-3-carboxylic acid (2-chloro-3-trifluoromethylbenzyl)cyclopropylamide;
4-{4-[2-(2-chloro-4-trifluoromethylphenoxy)ethoxy]phenyl}-1,2,5,6-tetrahydropyridine-3-carboxylic acid cyclopropyl-(2,3-dimethylbenzyl)amide;
4-{4-[2-(2,5-dichlorophenoxy)ethoxy]phenyl}-1,2,5,6-tetrahydropyridine-3-carboxylic acid (2-chloro-3-trifluoromethylbenzyl)cyclopropylamide;
4-{4-[2-(2,5-dichlorophenoxy)ethoxy]phenyl}-1,2,5,6-tetrahydropyridine-3-carboxylic acid cyclopropyl-(2,3-dimethylbenzyl)amide;
4-(4-[2-(2-chloro-4-trifluoromethylphenoxy)ethoxy]phenyl}-1,2,5,6-tetrahydropyridine-3-carboxylic acid cyclopropyl-(2,3-dichlorobenzyl)amide;
4-{4-[2-(2-bromo-5-fluorophenoxy)ethoxy]phenyl}-1,2,5,6-tetrahydropyridine-3-carboxylic acid cyclopropyl-(2,3-dichlorobenzyl)amide;
4-{4-[2-(2,3-dichlorophenoxy)ethoxy]phenyl}-1,2,5,6-tetrahydropyridine-3-carboxylic acid cyclopropyl-(2,3-dichlorobenzyl)amide;
4-{4-[2-(2-chloro-5-fluorophenoxy)ethoxy]phenyl-1,2,5,6-tetrahydropyridine-3-carboxylic acid cyclopropyl-(2,3-dichlorobenzyl)amide;
4-{4-[2-(2,5-dichlorophenoxy)ethoxy]phenyl}-1,2,5,6-tetrahydropyridine-3-carboxylic acid (2-bromobenzyl)cyclopropylamide;
4-{4-[2-(4-chloro-2-methylphenoxy)ethoxy]phenyl}-1,2,5,6-tetrahydropyridine-3-carboxylic acid cyclopropyl-(2,3-dichlorobenzyl)amide;
4-{4-[2-(2,6-dichloro-4-fluorophenoxy)ethoxy]phenyl}-1,2,5,6-tetrahydro-pyridine-3-carboxylic acid cyclopropyl-(3-trifluoromethoxybenzyl)amide;
4-{4-[2-(2,4,6-trifluorophenoxy)ethoxy]phenyl}-1,2,5,6-tetrahydropyridine-3-carboxylic acid cyclopropyl-(2-fluoro-5-methoxybenzyl)amide;
4-{4-[2-(2,6-dichloro-4-fluorophenoxy)ethoxy]phenyl}-1,2,5,6-tetrahydro-pyridine-3-carboxylic acid cyclopropyl-(3,5-dimethoxybenzyl)amide;
4-{4-[2-(2-chloro-4,5-dimethylphenoxy)ethoxy]phenyl}-1,2,5,6-tetrahydro-pyridine-3-carboxylic acid cyclopropyl-(2-fluoro-5-methoxybenzyl)amide;
4-{4-[2-(5-chloro-2-methoxyphenoxy)ethoxy]phenyl}-1,2,5,6-tetrahydro-pyridine-3-carboxylic acid (2-bromobenzyl)cyclopropylamide;
4-{4-[2-(2,3,6-trifluorophenoxy)ethoxy]phenyl}-1,2,5,6-tetrahydropyridine-3-carboxylic acid (2-bromobenzyl)cyclopropylamide;
4-{4-[2-(2-chloro-4,5-dimethylphenoxy)ethoxy]phenyl}-1,2,5,6-tetrahydro-pyridine-3-carboxylic acid cyclopropyl-(3,5-dimethoxybenzyl)amide;
4-{4-[2-(2-chloro-4,5-dimethylphenoxy)ethoxy]phenyl}-1,2,5,6-tetrahydro-pyridine-3-carboxylic acid (2-bromobenzyl)cyclopropylamide;
4-{4-[2-(2,6-dichloro-4-fluorophenoxy)ethoxy]phenyl}-1,2,5,6-tetrahydro-pyridine-3-carboxylic acid (3-chlorobenzyl)cyclopropylamide;
4-{4-[2-(2,6-difluoro-3-methylphenoxy)ethoxy]phenyl}-1,2,5,6-tetrahydro-pyridine-3-carboxylic acid cyclopropyl-(2,3-dimethoxybenzyl)amide;
4-{4-[2-(2,6-dichloro-4-fluorophenoxy)ethoxy]phenyl}-1,2,5,6-tetrahydro-pyridine-3-carboxylic acid cyclopropyl-(2-fluoro-5-methoxybenzyl)amide;
4-{4-[2-(2,6-difluoro-3-methylphenoxy)ethoxy]phenyl}-1,2,5,6-tetrahydro-pyridine-3-carboxylic acid (2-bromobenzyl)cyclopropylamide;
4-{4-[2-(2-fluorophenoxy)ethoxy]phenyl}-1,2,5,6-tetrahydropyridine-3-carboxylic acid (2-chloro-3-trifluoromethylbenzyl)cyclopropylamide;
4-{4-[2-(2,4,6-trifluorophenoxy)ethoxy]phenyl}-1,2,5,6-tetrahydropyridine-3-carboxylic acid (6-chlorobenzo[1,3]dioxol-5-ylmethyl)cyclopropylamide;
4-{4-[2-(3-chloro-2,6-difluorophenoxy)ethoxy]phenyl}-1,2,5,6-tetrahydro-pyridine-3-carboxylic acid (6-chlorobenzo[1,3]dioxol-5-ylmethyl)-cyclopropylamide;
4-{4-[2-(2,6-dichloro-4-methylphenoxy)ethoxy]phenyl}-1,2,5,6-tetrahydro-pyridine-3-carboxylic acid cyclopropyl-(3,5-difluorobenzyl)amide;
4-{4-[2,4,5-trichlorophenoxy)ethoxy]phenyl}-1,2,5,6-tetrahydropyridine-3-carboxylic acid (2-chloro-3-trifluoromethylbenzyl)cyclopropylamide;
4-{4-[2-(2-chloro-5-fluorophenoxy)ethoxy]phenyl}-1,2,5,6-tetrahydropyridine-3-carboxylic acid (2-chloro-3-trifluoromethylbenzyl)cyclopropylamide;
4-{4-[2-(2,3-dichlorophenoxy)ethoxy]phenyl}-1,2,5,6-tetrahydropyridine-3-carboxylic acid cyclopropyl-(2,3-dimethylbenzyl)amide;
4-{4-[2-(2,6-dichloro-4-fluorophenoxy)ethoxy]phenyl}-1,2,5,6-tetrahydro-pyridine-3-carboxylic acid (2-chlorobenzyl)ethylamide;
4-{4-[2-(2,4,5-trichlorophenoxy)ethoxy]phenyl}-1,2,5,6-tetrahydropyridine-3-carboxylic acid cyclopropyl-(2,3-dimethylbenzyl)amide;
4-{4-[2-(3-chloro-2,6-difluorophenoxy)ethoxy]phenyl}-1,2,5,6-tetrahydro-pyridine-3-carboxylic acid (2-bromobenzyl)cyclopropylamide;
4-{4-[2-benzol[1,3]dioxol-5-yloxy)ethoxy]phenyl}-1,2,5,6-tetrahydro-pyridine-3-carboxylic acid cyclopropyl-(2,3-dimethylbenzyl)amide;
4-{4-[2-(2-chloro-4-trifluoromethylphenoxy)ethoxy]phenyl}-1,2,5,6-tetrahydro-pyridine-3-carboxylic acid cyclopropyl-(3,5-dimethoxybenzyl)amide;
4-{4-[2-(2,4,6-trifluorophenoxy)ethoxy]phenyl}-1,2,5,6-tetrahydropyridine-3-carboxylic acid cyclopropyl-(3,5-dimethoxybenzyl)amide;
4-{4-[2-(2,4,6-trifluorophenoxy)ethoxy]phenyl}-1,2,5,6-tetrahydropyridine-3-carboxylic acid (2-chlorobenzyl)ethylamide;
4-{4-[2-(2-chloro-4-trifluoromethylphenoxy)ethoxy]phenyl-1,2,5,6-tetrahydropyridine-3-carboxylic acid cyclopropyl-(2-fluoro-5-methoxy-benzyl)amide;
4-{4-[2-(2,6-dichloro-4-fluorophenoxy)ethoxy]phenyl}-1,2,5,6-tetrahydro-pyridine-3-carboxylic acid (6-chlorobenzo[1,3]dioxol-5-ylmethyl)-cyclopropylamide;
4-{4-[2-(4-chloro-2-methoxyphenoxy)ethoxy]phenyl}-1,2,5,6-tetrahydro-pyridine-3-carboxylic acid cyclopropyl-(3-methoxybenzyl)amide;
4-{4-[2-(2-chloro-4-trifluoromethylphenoxy)ethoxy]phenyl}-1,2,5,6-tetrahydropyridine-3-carboxylic acid (2-bromobenzyl)cyclopropylamide;
4-{4-[2-(2-chloro-4-trifluoromethylphenoxy)ethoxy]phenyl}-1,2,5,6-tetrahydro-pyridine-3-carboxylic acid cyclopropyl-(3-methoxybenzyl)amide;
4-{4-[2-(2,3,6-trifluorophenoxy)ethoxy]phenyl}-1,2,5,6-tetrahydropyridine-3-carboxylic acid (2-chlorobenzyl)ethylamide;
4-{4-[2-(3-chloro-2,6-difluorophenoxy)ethoxy]phenyl}-1,2,5,6-tetrahydro-pyridine-3-carboxylic acid cyclopropyl-(2-fluoro-5-methoxybenzyl)amide;
4-{4-[2-(2-bromo-5-fluorophenoxy)ethoxy]phenyl}-1,2,5,6-tetrahydropyridine-3-carboxylic acid cyclopropyl-(2-fluoro-5-methoxybenzyl)amide;
4-{4-[2-(2,5-dichlorophenoxy)ethoxy]phenyl}-1,2,5,6-tetrahydropyridine-3-carboxylic acid cyclopropyl-(3,5-dimethoxybenzyl)amide;
4-{4-[2-(2-chloro-4,5-dimethylphenoxy)ethoxy]phenyl}-1,2,5,6-tetrahydro-pyridine-3-carboxylic acid cyclopropyl-(3-methoxybenzyl)amide;
4-{4-[2-(4-chloro-2-methylphenoxy)ethoxy]phenyl}-1,2,5,6-tetrahydropyridine-3-carboxylic acid cyclopropyl-(2,3-dimethylbenzyl)amide
4-{4-[2-(4-chloro-2-methylphenoxy)ethoxy]phenyl}-1,2,5,6-tetrahydro-pyridine-3-carboxylic acid (6-chlorobenzo[1,3]dioxol-5-ylmethyl)-cyclopropylamide;
4-{4-[2-(2-bromo-5-fluorophenoxy)ethoxy]phenyl}-1,2,5,6-tetrahydropyridine-3-carboxylic acid (2-bromobenzyl)cyclopropylamide;
4-{4-[2-(3-chloro-2,6-difluorophenoxy)ethoxy]phenyl}-1,2,5,6-tetrahydro-pyridine-3-carboxylic acid cyclopropyl-(3-methoxybenzyl)amide;
4-{4-[2-(2-chloro-4-trifluoromethylphenoxy)ethoxy]phenyl}-1,2,5,6-tetrahydro-pyridine-3-carboxylic acid (6-chlorobenzo[1,3]dioxol-5-ylmethyl)-cyclopropylamide;
4-{4-[2-(2-chloro-4,5-dimethylphenoxy)ethoxy]phenyl}-1,2,5,6-tetrahydro-pyridine-3-carboxylic acid (2-chlorobenzyl)cyclopropylamide;
4-4-[2-(2,6-dichloro-4-methylphenoxy)ethoxy]phenyl}-1,2,5,6-tetrahydro-pyridine-3-carboxylic acid (2-chlorobenzyl)cyclopropylamide;
4-{4-[2-(2,4,5-trichlorophenoxy)ethoxy]phenyl}-1,2,5,6-tetrahydropyridine-3-carboxylic acid (2-chlorobenzyl)cyclopropylamide;
4-{4-[2-(2-chloro-5-fluorophenoxy)ethoxy]phenyl}-1,2,5,6-tetrahydropyridine-3-carboxylic acid (2-chlorobenzyl)cyclopropylamide;
4-{4-[2-(2-chloro-3,6-difluorophenoxy)ethoxy]phenyl}-1,2,5,6-tetrahydro-pyridine-3-carboxylic acid (2-chlorobenzyl)cyclopropylamide;
4-(4-[2-(2-chloro-6-methylphenoxy)ethoxy]phenyl}-1,2,5,6-tetrahydropyridine-3-carboxylic acid (2-chlorobenzyl)cyclopropylamide;
4-{4-[2-(2,3-dichlorophenoxy)ethoxy]phenyl}-1,2,5,6-tetrahydro-pyridine-3-carboxylic acid (2-chlorobenzyl)cyclopropylamide;
4-{4-[2-(2,6-dichloro-4-fluorophenoxy)ethoxy]phenyl}-1,2,5,6-tetrahydro-pyridine-3-carboxylic acid (2-chlorobenzyl)cyclopropylamide;
4-(4-[2-(3-chloro-2,6-difluorophenoxy)ethoxy]phenyl}-1,2,5,6-tetrahydropyridine-3-carboxylic acid (2-chlorobenzyl)cyclopropylamide;
4-{4-[2-(2,4,6-trifluorophenoxy)ethoxy]phenyl}-1,2,5,6-tetrahydropyridine-3-carboxylic acid (2-chlorobenzyl)cyclopropylamide;
4-{4-[2-(2,5-dichlorophenoxy)ethoxy]phenyl}-1,2,5,6-tetrahydropyridine-3-carboxylic acid (2-chlorobenzyl)cyclopropylamide; and
4-{4-[2-(2,6-dichlorophenoxy)ethoxy]phenyl}-1,2,5,6-tetrahydropyridine-3-carboxylic acid (2-chlorobenzyl)cyclopropylamide.

7. A a compound of any one of claims 1-6 and a pharmaceutically acceptable carrier with or without an adjuvant.

8. A method for the treatment or prophylaxis of a disease associated with dysregulation of the rennin-angiotensin system (RAS), comprising administrating a compound according to any one of claims 1 to 6 to a subject in need thereof.

9. (canceled)

10. (canceled)

11. The method of claim 8, wherein said disease is selected from the group consisting of hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, and complications of treatment with immunosuppressive agents after organ transplantation.

12. The pharmaceutical composition of claim 7, further comprising an additional pharmacologically active compound selected from the group consisting of ACE inhibitors, angiotensin II receptor antagonists, endothelin receptor antagonists, vasodilators, calcium antagonists, potassium activators, diuretics, sympatholitics, beta-adrenergic antagonists and alpha-adrenergic antagonists.

13. A method for treating or prevemtomg a disease associated with dysregulation of the RAS, comprising administering the pharmaceutical composition of claim 7 to a subject in need thereof.

14. A method for treating or preventing a disease associated with dysregulation of the RAS, comprising administering the pharmaceutical composition of claim 12 to a subject in need thereof.
